(19) United States
(12) Patent Application Publication
(10) Pub. No.: US 2025/0361288 A1
(43) Pub. Date: Nov. 27, 2025

(54) LIGHT MUTEINS AND USES THEREOF

(71) Applicant: Gilead Sciences, Inc., Foster City, CA (US)

(72) Inventors: Qianting ZHAI, South San Francisco, CA (US); Maiyun YANG, Shanghai (CN); Mingjian FEI, Newark, CA (US); Xiaodong HUANG, Belmont, CA (US)

(21) Appl. No.: 18/872,726

(22) PCT Filed: Jun. 8, 2023
(86) PCT No.: PCT/CN2023/099158

§ 371 Date: Dec. 6, 2024

(30) Foreign Application Priority Data
Jun. 8, 2022 (WO) PCT/CN2022/097735

(51) Int. Cl. C07K 14/705; A61K 38/17

(52) U.S. Cl. CPC C07K 14/70575; A61K 38/177

(57) ABSTRACT

The present disclosure provides a LIGHT mutein and a LTβR binding LIGHT mutein, and it also provides a related polynucleotide, an isolated vector, a host cell, and a pharmaceutical composition. Further, the present disclosure provides the use of the LIGHT mutein or the isolated polynucleotide, the isolated vector, the host cell, or the pharmaceutical composition in the manufacture of a drug for preventing or treating a disease, and a method of preventing or treating a disease in a subject in need thereof.

### CROSS-REFERENCE TO RELATED APPLICATIONS

This application is a U.S. National Stage Application under 35 U.S.C. § 371 of International Patent Application No. PCT/CN2023/099158, filed Jun. 8, 2023, which claims the benefit of priority to International Patent Application No. PCT/CN2022/097735, filed on Jun. 8, 2022, the entire contents of which are each considered as a part of the present disclosure and are incorporated herein by reference.

### REFERENCE TO THE SEQUENCE LISTING

The Sequence Listing titled 210196-348003US_SL.xml, which was created on Dec. 5, 2024 and is 95,560 bytes in size, is hereby incorporated by reference in its entirety.

### TECHNICAL FIELD

The present disclosure relates to a LIGHT mutein (or “LIGHT mutant”), an isolated vector comprising the polynucleotide encoding the LIGHT mutein, a host cell comprising the isolated polynucleotide or the isolated vector encoding the LIGHT mutein, a pharmaceutical composition comprising the LIGHT mutein/muteins, the use of the LIGHT mutein, the isolated polynucleotide, the isolated vector, the host cell or the pharmaceutical composition in the manufacture of a drug for preventing or treating a disease, and a method of preventing or treating a disease in a subject in need thereof, comprising administrating to the subject a therapeutically effective amount of the LIGHT mutein/muteins or the isolated polynucleotide, the isolated vector, the host cell or the pharmaceutical composition.

### BACKGROUND

The documentation in this section is only for the purpose of providing the background information related to the present disclosure, and the information present under this section does not necessarily constitute the prior art.

LIGHT (Lymphotoxin-like, exhibits inducible expression and competes with Herpes Simplex Virus glycoprotein D for Herpes Virus Entry Mediator, a receptor expressed by T cells) is known as tumor necrosis factor superfamily member 14 (TNFSF14), also referred to as HVEM-Ligand (HVEM-L). LIGHT is a membrane protein composed of 240 amino acids (AAs) (SEQ ID NO:86), of which 37 AAs form the cytoplasmic domain, 22 AAs form the transmembrane domain, and 181 AAs form the extracellular domain. LIGHT is transiently induced on the immune cells, especially the immature dendritic cells (DCs) and the activated T cells. The membrane-anchored form of LIGHT can be cleaved by proteases, resulting in a soluble functional structure (Yu et al., 2004).

LIGHT has three receptors: herpes virus entry mediator (HVEM), lymphotoxin beta receptor (LTβR), and decoy receptor 3 (DcR3). HVEM is expressed on T cells, NK cells and dendritic cells. The interaction between LIGHT and HVEM stimulates T cell activation, proliferation and survival. Another receptor LTβR is found on the surface of epithelial, stromal, immature DCs, and myeloid cells, but not on the lymphocytes. The LIGHT-LTβR interaction leads to the expression of chemokines and adhesion molecules involved in lymph node formation and dendritic cell migration. The third binding partner, DcR3, is a soluble protein, which dampens the activation signal initiated by LIGHT (Liu et al., 2021). Introducing LIGHT into tumors or tumor microenvironment could be a potent strategy for cancer immunotherapy.

We have engineered human LIGHT to selectively interact with its receptors aiming to utilize the recombinant LIGHT protein as a therapeutic agent.

### SUMMARY

For the above-mentioned purpose, provided herein is a novel LIGHT mutein. In some embodiments, the LIGHT muteins have the sequence set forth in SEQ ID NO: 87 or SEQ ID NO:88 or SEQ ID NOs: 1-85, or SEQ ID NOs: 89-93.

In some embodiments, provided herein is a LIGHT mutein having more than 80%, 85%, 90%, 95%, 96%, 97%, 98%, 99% homology to the sequence set forth in SEQ ID NO: 86. In some embodiments, provided herein is a LIGHT mutein having more than 80%, 85%, 90%, 95%, 96%, 97%, 98%, 99% homology to the sequence set forth in SEQ ID NO: 87. In some embodiments, provided herein is a LIGHT mutein having more than 80%, 85%, 90%, 95%, 96%, 97%, 98%, 99% homology to the sequence set forth in SEQ ID NO: 88. In some embodiments, provided herein is a LIGHT mutein having more than 80%, 85%, 90%, 95%, 96%, 97%, 98%, 99% homology to the sequence set forth in SEQ ID NOs: 1-85 or SEQ ID NOs: 89-93.

Provided herein is a novel LIGHT mutein, which is selected from the group consisting a protein having more than 80%, 85%, 90%, 95%, 96%, 97%, 98%, 99% homology to the sequence set forth in SEQ ID NO: 87. In some embodiments, the novel LIGHT muteins, comprising one or more amino acid mutations as compared with the amino acid sequence set forth in SEQ ID NO: 86. The amino acid mutations selected from one or more positions selected from the group consisting of 95, 103, 117, 125, 150, 152, 155, 157, 158, 160, 161, 175, 184, 189, 190, 198, 202, 208, 214, 220, 221, 227, 228, and the combination of any of them, wherein the positions are defined with reference to SEQ ID NO: 86.

In some embodiments, the LIGHT muteins comprise one or more amino acid mutations as compared with the amino acid sequence set forth in SEQ ID NO: 87. The amino acid mutations selected from one or more positions selected from the group consisting of 95, 103, 117, 125, 150, 152, 155, 157, 158, 160, 161, 175, 184, 189, 190, 198, 202, 208, 214, 220, 221, 227, 228, and the combination of any of them, wherein the positions are defined with reference to SEQ ID NO: 87, and the position of the first amino acid of SEQ ID NO: 87 is defined as position 74.

In some embodiments, the LIGHT muteins comprise one or more amino acid mutations as compared with the amino acid sequence set forth in SEQ ID NO: 88. The amino acid mutations selected from one or more positions selected from the group consisting of 95, 103, 117, 125, 150, 152, 155, 157, 158, 160, 161, 175, 184, 189, 190, 198, 202, 208, 214, 220, 221, 227, 228, and the combination of any of them, wherein the positions are defined with reference to SEQ ID NO: 88, and the position of the first amino acid of SEQ ID NO: 88 is defined as position 87.

In some embodiments, provided herein is a LIGHT mutein having the sequence set forth in SEQ ID No. 86, 87 or 88 and having one or more amino acid mutations selected from the group consisting of S103N, Q117E/Q117N/Q117H/Q117R, L126M, G150A/G150S/G150R, V152M, L158Q/L158P/L158M, S160G/S160N/S160T/S160A, T161G/T161P/T161S/T161N, L166M, E175K, Q184R, R189S, A190T/A190V, W198Q, F202Y, H208Y/H208R, K214E, L220S/L220N/L220T/L220R/L220M, D221G, E222K/E222S, L227T/L227M, R228L, R232H and the combination of any of them.

In some embodiments, provided herein is a LIGHT mutein having the sequence set forth in SEQ ID No. 100 and having one or more amino acid mutations selected from the group consisting of A95T, A101D, N102R, S103N, Q117E/Q117N/Q117H/Q117R, L126M, V135I, T136S, G150A/G150S/G150R, V152M, P155R, G157S, L158Q/L158P/L158M, S160G/S160N/S160T/S160A, T161G/T161P/T161S/T161N, L166M, P174L, E175K, Q184R, R189S, A190T/A190V, W198Q, F202Y, H208Y/H208R, E213D, K214E, L220S/L220N/L220T/L220R/L220M, D221G, E222K/E222S/E222Q/E222D/E222N, L227T/L227M, R228L, R232H and the combination of any of them, wherein the positions are defined with reference to SEQ ID NO: 87, and the position of the first amino acid of SEQ ID NO: 87 is defined as position 74.

In some embodiments, provided herein is a LIGHT mutein having the sequence set forth in SEQ ID No. 88 and having one or more amino acid mutations selected from the group consisting of A95T, A101D, N102R, S103N, S104P, L105P, T116S, Q117E/Q117N/Q117H/Q117R, L126M, V135I, T136S, G150A/G150S/G150R, V152M, P155R, L156P, G157S, L158Q/L158P/L158M, S160G/S160N/S160T/S160A, T161G/T161P/T161S/T161N, L166M, P174L, E175K, Q184R, R189S, A190T/A190V, W198Q, F202Y, H208Y/H208R, E213D, K214E, L220S/L220N/L220T/L220R/L220M, D221G, E222K/E222S/E222Q/E222D/E222N, L227T/L227M, R228L, R232H and the combination of any of them, wherein the positions are defined with reference to SEQ ID NO: 88, and the position of the first amino acid of SEQ ID NO: 87 is defined as position 87.

In some embodiments, provided herein is an LTβR binding LIGHT mutein, which is selected from the group consisting of LIGHT muteins having the sequence set forth in SEQ ID NOs: 1-75, 76-85 and SEQ ID NOs: 89-93. Or the mutein is selected from a protein which having the sequence set forth in SEQ ID NO: 87 or SEQ ID NO:88 and compared with SEQ ID NO: 87 or SEQ ID NO:88 having at least one amino acid difference.

In some embodiments, the LTβR binding LIGHT mutein is HVEM (e.g., human HVEM) non-binding, the LIGHT mutein includes the amino acid sequence set forth in any one of SEQ ID NOs: 1-11.

In some embodiments, the LTβR binding LIGHT mutein is HVEM binding, the LIGHT mutein includes the amino acid sequence set forth in any one of SEQ ID NOs: 12-75, SEQ ID NOs: 77-85 and SEQ ID NOs: 89-93.

In some embodiments, the LTβR binding LIGHT mutein binds to DcR3 with reduced affinity, compared to wildtype LIGHT, the LIGHT mutein includes an amino acid sequence set forth in any one of SEQ ID NOs: 1, 2, 52, 58, 61 and 89-93.

In some embodiments, provided herein is an LTβR binding LIGHT mutein, which is an mHVEM (mouse HVEM) binding protein and hHVEM (human HVEM) non-binding protein. For example, the sequence set forth in SEQ ID NOs: 1, 2, 9 or 11.

In some embodiments, provided herein is a human LTβR binding LIGHT mutein, which is selected from the group consisting of LIGHT mutein having the sequence set forth in SEQ ID NOs: 1, 2, 9, 11, 12, 22, 37, 52. 53-85.

In some embodiments, provided herein is a mouse LTβR binding LIGHT mutein, which is selected from the group consisting of LIGHT mutein having the sequence set forth in SEQ ID NOs: 1, 9, 11, 12, 22, 38 and 52. In some embodiments, provided herein is an LTβR binding and hHVEM binding LIGHT mutein, which is selected from the group consisting of LIGHT muteins having the sequence set forth in SEQ ID NOs: 21, 22, 37 and 51. In some embodiments, provided herein is an LTβR binding and hHVEM non-binding LIGHT mutein, which is selected from the group consisting of LIGHT muteins having the sequence set forth in SEQ ID NOs: 1, 2, 9 and 11.

In some embodiments, provided herein is an LTβR binding LIGHT in truncated form and the muteins thereof comprising the amino acid sequence set forth in any one of SEQ ID NOs: 1 to 85. In some embodiments, provided herein is an LTβR binding and mHVEM binding LIGHT in truncated form and the muteins thereof, comprising the amino acid sequence set forth in any one of SEQ ID NOS 1 to 85. In some embodiments, provided herein is an LTβR binding and mHVEM binding LIGHT in truncated form and the muteins thereof, which is hHVEM non-binding, comprising the amino acid sequence set forth in any one of SEQ ID NOs: 1-11.

In some embodiments, provided herein is an LTβR binding and HVEM binding LIGHT muteins, comprising the amino acid sequence set forth in any one of SEQ ID NOs: 12-75, SEQ ID NOs: 77-85 and SEQ ID NOs: 89-93.

In another aspect, provided herein is an isolated polynucleotide encoding the LIGHT mutein provided herein.

In another aspect, provided herein is an isolated vector comprising the polynucleotide encoding the LIGHT mutein.

In another aspect, provided herein is a host cell comprising the isolated polynucleotide or the isolated vector encoding the LIGHT mutein.

In another aspect, provided herein is a pharmaceutical composition comprising the LIGHT mutein/muteins, the isolated polynucleotide, the isolated vector or the host cell.

In another aspect, provided herein is the use of the LIGHT mutein or the isolated polynucleotide or the isolated vector or the host cell or the pharmaceutical composition in the manufacture of a therapeutic agent for diagnosing, preventing or treating a disease disorder, or condition.

The disclosure provides a method of diagnosing, preventing or treating a disease, disorder, or condition in a subject in need thereof, comprising administrating to the subject a therapeutically effective amount of the LIGHT mutein/muteins, the isolated polynucleotide, the isolated vector, the host cell, or the pharmaceutical composition.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows the ELISA analysis of LIGHT muteins binding to the human LTβR extracellular domain. LIGHT-9, LIGHT-11, LIGHT-21, LIGHT-22, LIGHT-52, LIGHT-3, LIGHT-29 and LIGHT-20 show stronger binding affinities than wild-type human LIGHT. The X-axis is the concentration of LIGHT protein (ng/ml), and the Y-axis is the absorbance at 450 nm.

FIG. 2 shows the ELISA analysis of LIGHT muteins binding to the mouse LTβR extracellular domain. LIGHT-3, LIGHT-9, LIGHT-11, LIGHT-29, LIGHT-22 and LIGHT-52 show stronger binding affinity than wild-type human LIGHT. The X-axis is the concentration of LIGHT protein (ng/ml), and the Y-axis is the absorbance at 450 nm.

FIG. 3 shows the ELISA analysis of LIGHT muteins binding to the human HVEM extracellular domain. LIGHT-20, LIGHT-21, LIGHT-22, LIGHT-29 and LIGHT-52 show stronger binding affinity than wild-type human LIGHT, while LIGHT-3, LIGHT-9 and LIGHT-11 do not bind to human HVEM. The X-axis is the concentration of LIGHT protein (ng/ml), and Y-axis is the absorbance at 450 nm.

FIGS. 4A and 4B show the alignment results of amino acids 1-60 and 61-120 of LIGHT-1 to LIGHT-90 with full-length human LIGHT, respectively.

FIGS. 5A and 5B show the alignment results of amino acids 121-180 and 181-240 of LIGHT-1 to LIGHT-90 with full-length human LIGHT, respectively.

FIG. 6 shows the ELISA analysis of LIGHT muteins binding to the human LTβR extracellular domain. LIGHT-1, LIGHT-2, LIGHT-58, LIGHT-52, LIGHT-86 and LIGHT-88 show similar binding affinity with wild-type human LIGHT. The X-axis is the concentration of trimeric LIGHT protein (nM), and the Y-axis is the absorbance at 450 nm.

FIG. 7 shows the ELISA analysis of LIGHT muteins binding to the mouse LTβR extracellular domain. LIGHT-58, LIGHT-60, LIGHT-61, LIGHT-86, LIGHT-87, LIGHT-88 and LIGHT-89 show stronger binding affinity than wild-type human LIGHT. The X-axis is the concentration of trimeric LIGHT protein (nM), and the Y-axis is the absorbance at 450 nm.

FIG. 8 shows the ELISA analysis of LIGHT muteins binding to the human HVEM extracellular domain. LIGHT-58, LIGHT-60, LIGHT-61 and LIGHT-52 show stronger binding affinity than wild-type human LIGHT, while LIGHT-1, LIGHT-2, LIGHT-88 and LIGHT-89 do not bind to human HVEM. The X-axis is the concentration of trimeric LIGHT protein (nM), and the Y-axis is the absorbance at 450 nm.

FIG. 9 shows the ELISA analysis of LIGHT muteins binding to the human DcR3 (Uniprot 095407). LIGHT-1, LIGHT-2, LIGHT-86, LIGHT-87, LIGHT-88, LIGHT-89 and LIGHT-90 show reduced binding affinity than wild-type human LIGHT. The X-axis is the concentration of LIGHT protein (nM), and the Y-axis is the absorbance at 450 nm.

FIG. 10 shows the ELISA analysis of LIGHT-60, LIGHT-61 and LIGHT-90 binding to the human LTβR extracellular domain. LIGHT-60, LIGHT-61 and LIGHT-90 show similar affinity to human LTβR extracellular domain, compared to LIGHT (74-240) (LIGHTwt, LIGHT wild type ECD). The X-axis is the concentration of LIGHT protein (nM), and the Y-axis is the absorbance at 450 nm.

FIG. 11 shows the functional activation of human LTβR by LIGHT90, LIGHT60 and LIGHT63 in Hela-NK-kB-reporter cells. The EC50s of each variant are shown in the table.

### DETAILED DESCRIPTION

The present disclosure is explained in greater details below. This description is not intended to be a detailed catalog of all the different ways in which the invention may be implemented or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

### Definitions

The term “naturally occurring” as used herein refers to a sequence of natural origin which means that the whole or parts thereof are not synthetic and exist or are produced in nature. More preferably, the term “naturally occurring” as used herein refers to a sequence of natural origin which means that the whole sequence is not synthetic and exists or is produced in nature.

The term “mutated”, “mutation”, “mutein” and “mutant” are interchangeably used herein. Typically, and preferably, a mutation is a substitution of one amino acid by one or more amino acids, an insertion, a deletion or a combination thereof. More preferably, a mutation is a substitution of a single amino acid by a different single amino acid.

The term “LIGHT” has the meaning commonly understood in the art and refers to a protein expressed on activated CD4/CD8 T cells, dendritic cells (DCs), monocytes, and natural killer cells (NK). The binding of LIGHT to herpes virus entry mediator (HVEM) expressed on resting T cells, DCs, and monocytes, or to the lymphotoxin beta receptor (LTβR) expressed on DCs and stromal cells promotes T cell activation, proliferation, and cytokine production. The entire amino acid sequence of LIGHT is shown in SEQ ID NO:86.

As used herein, “LIGHT mutein” means the muteins derived from the sequence set forth in SEQ ID NO: 86 with the mutation in one or more amino acids, said mutation is a substitution of one amino acid by one or more amino acids, an insertion, a deletion or a combination thereof. More preferably, said mutation is a substitution of a single amino acid by a different single amino acid. The LIGHT muteins at least comprise the sequence shown in SEQ ID NO:87 and have one or more amino acids substituted by a different single amino acid.

As used herein, “LIGHT mutein” also includes “LIGHT mutein in truncated form”, “LIGHT mutein in truncated form” refers to a shorter LIGHT, comparing with naturally occurring LIGHT shown in SEQ ID NO:86, which covers a main functional region of LIGHT without transmembrane domain of LIGHT, as the example used herein, LIGHT mutein in a truncated form comprising the sequences of LIGHT 74-240 (SEQ ID NO:87), LIGHT (87-240) (SEQ ID NO:88) and LIGHT muteins (SEQ ID NOs: 1-85), or the combination thereof.

“LTβR” means lymphotoxin beta receptor.

“mLTβR” means lymphotoxin beta receptor derived from mouse, e.g., Uniport P50284.

“hLTβR” means lymphotoxin beta receptor sourced from human, e.g., Uniport P36941.

“LTβR binding LIGHT mutein” means LIGHT mutein proteins which can bind to LTβR.

“HVEM” means herpes virus entry mediator.

“mHVEM” means herpes virus entry mediator derived from mouse, e.g., Uniport Q80WM9.

“hHVEM” means herpes virus entry mediator sourced from human, e.g., Uniport Q92956.

“LTβR binding and mHVEM binding LIGHT mutein” means LIGHT mutein proteins which can bind to both LTβR and mouse HVEM.

### LIGHT Muteins

The present disclosure provides a LIGHT mutein, which is at least 80%, 85%, 90%, 95%, 96%, 97%, 98%, 99% identical to an amino acid sequence set forth in SEQ ID NO: 86, 87 or 88.

In some embodiments, the LIGHT mutein provided herein is at least 80%, 85%, 90%, 95%, 96%, 97%, 98%, 99% identical to an amino acid sequence set forth in SEQ ID NO: 88.

The homology to a sequence is well known to those skilled in the art. The method to measure the homology to a sequence, including, but not limited to, BLAST on website of NCBI.

The LIGHT mutein provided herein includes at least one amino acid mutation compared with the amino acid sequence set forth in SEQ ID NO: 86, 87 or 88.

In some embodiments, the LIGHT mutein includes the amino acid mutations at positions selected from the group consisting of 95, 101, 102, 103, 104, 116, 117, 120, 126, 135, 136, 150, 152, 155, 156, 157, 158, 160, 161, 166, 174, 175, 184, 189, 190, 198, 202, 208, 213, 214, 220, 221, 222, 223, 227, 228, 232, and the combination thereof, wherein the positions are defined with reference to SEQ ID NO: 87.

The position of the first amino acid of SEQ ID NO: 87 is defined as position 74.

In some embodiments, the LIGHT mutein includes one or more amino acid mutations selected from the group consisting of A95T, A101D, N102R, S103N, S104P, L105P, T116S, Q117E/Q117N/Q117H/Q117R, L120P/L120Q, L126M, V135I, T136S, G150A/G150S/G150R, V152M, P155R, L156P, G157S, L158Q/L158P/L158M, S160G/S160N/S160T/S160A/S160H/S160R, T161G/T161P/T161S/T161N, L166M, P174L, E175K, Q184R, R189S, A190T/A190V, W198Q, F202Y, H208Y/H208R, E213D, K214E, L220S/L220N/L220T/L220R/L220M/L220Q, D221G, E222K/E222S/E222Q/E222D/E222N, R223H, L227T/L227M, R228L, R232H and the combination thereof, compared to amino acid sequence set forth in SEQ ID NO: 87.

In the present disclosure, the mutation “A95T” means that the amino acid A at position 95 is mutated to amino acid T. The mutation “L120P/L120Q” means mutation L120P or L120Q. Other mutations described in the present disclosure have the similar meaning.

In the present disclosure, the amino acid is presented as standard single-letter code according to the standard IUPAC (International Union of Pure and Applied Chemistry) amino acid abbreviation.

In some embodiments, the LIGHT mutein includes one or more amino acid mutations selected from Q117E/Q117N/Q117H, G150A/G150S, S160G/S160N/S160T/S160H/S160A, T161G/T161P/T161S/T161N, W198Q, K214E, L220S/L220N/L220T/L220M/L220R/L220Q, E222K/E222S/E222Q/E222D/E222N, R228L and R232H.

In some embodiments, the LIGHT mutein includes one or more amino acid mutations selected from A95T, A101D, N102R, S103N, S104P, L105P, T116S, Q117E/Q117N/Q117H/Q117R, L126M, V135I, T136S, G150A/G150S/G150R, V152M, P155R, L156P, G157S, L158Q/L158P/L158M, S160G/S160N/S160T/S160A, T161G/T161P/T161S/T161N, L166M, P174L, E175K, Q184R, R189S, A190T/A190V, W198Q, F202Y, H208Y/H208R, E213D, K214E, L220S/L220N/L220T/L220R/L220M, D221G, E222K/E222S/E222Q/E222D/E222N, L227T/L227M, R228L and R232H.

In some embodiments, the LIGHT mutein includes any one of mutation combinations: V152M, W198Q and R228L; R189S, W198Q and R228L; G150A and L220S; V152M, T161P and R228L; T161P, R189S, W198Q and R228L; W198Q, L220N and D221G; P155R, L220Q and R232H; G150S, S160G and L220S; G150S, T161G and L220S; G150S, T161P and L220S; G150S and L220S; L158Q and K214E; S104P, G157S, H208Y and L220R; L158Q and L166M; Q117E, E175K, K214E and L227T; Q117H, L158M and E213D; H208Y and Q117N; L158Q, K214E and E222K; L156P, S160G and L220M; S160A, L220S and E222D; S160G, T161P and L220T; S160G, T161S and L220S; S160G and L220S; S160G, A190T and L220S; S160H and L220S; S160H, L220S and E222Q; S160N, T161S, L220S and E222D; S160N, K214E and L227M; S160N and L220S; S160R, T161N and L220S; S160R, T161S and L220S; S160R and L220S; T161P and L220S; T161S and L220S; T161S, L220S and E222N; H208Y; A190T, F202Y and K214E; Q184R and W198Q; L158P, L166M and L220M; L156P and H208Y; G150R, S160G and L220S; G150R and L220S; G150R, S160T and L220S; G150R, L158P and L220S; G150S, S160P, T161S, L220S and R223H; L126M and K214E; L126M, A190V and H208Y; L120P and L220R; L105P, Q117R and L220Q; N102R, L120Q, H208R and K214E; A101D, S160N and L220S; L220S; Q117N, L220S and L227T; Q117E, L220S and L227T; Q117N, L220T and L227T; Q117E, L220T and L227T; L220S and L227T; S160G, T161G and L220S; S160G, T161P and L220S; S160G, T161G, L220S and E222S; S160G, T161P, L220S and E222S; S160G, T161G and L220T; T161G, L220S and L227T; T161P, L220S and L227T; S160G, L220S and L227T; S160G, L220T and L227T; T161S, L220S and L227T; S160G, T161G, L220S and L227T; S160G, T161P, L220S and L227T; Q117N, T161G, L220S and L227T; Q117N, T161P, L220S and L227T; Q117N, S160G, L220S and L227T; Q117E, T161G, L220S and L227T; Q117N, S160G, T161G, L220S and L227T; Q117N, S160G, T161P, L220S and L227T; Q117E, S160G, T161P, L220S and L227T; Q117N, S160G, T161S, L220S and L227T; Q117E, S160G, T161S, L220S and L227T; Q117N, S160G, T161G, L220S, E222S and L227T; Q117N, S160G, T161P, L220S, E222S and L227T; Q117E, S160G, T161G, L220S, E222S and L227T; Q117E, S160G, T161P, L220S, E222S and L227T; S160G, T161S, L220S, E222S and L227T; Q117E, S160G, T161S, L220S, E222S and L227T; G150R, S160R and L220S; G150R, S160R, W198Q, L220S and R228L; A95T, G150R, S160R, P174L, W198Q, L220S and R228L; G150R, S160R, P174L, W198Q and R228L; or S160G, T161G, L220T and E222S.

In some embodiments, the LIGHT mutein has an amino acid sequence with at least 95%, 96%, 97%, 98% or 99% homology to amino acid sequence set forth in any one of SEQ ID NOs: 1 to 75, 77 to 85 and SEQ ID NOs: 89 to 93. In some embodiments, the LIGHT mutein has an amino acid sequence set forth in any one of SEQ ID NOs: 1 to 75, 77 to 85 and SEQ ID NOs: 89 to 93.

In some embodiments, the LIGHT mutein has an amino acid sequence that is different from any one sequence of SEQ ID NOs: 1 to 75, 77 to 85 and SEQ ID NOs: 89 to 93 by no more than 10, 9, 8, 7, 6, 5, 4, 3, 2 or 1 amino acid.

The LIGHT muteins selectively bind to LIGHT receptors, LTβR, HVEM or DcR3. In some embodiment, the LIGHT muteins are LTβR binding. In some embodiment, the LIGHT muteins are HVEM binding. In some embodiment, the LIGHT muteins are HVEM non-binding.

In some embodiment, compared to wildtype LIGHT, the LIGHT muteins bind to LTβR with improved affinity. In some embodiment, compared to wildtype LIGHT, the LIGHT muteins bind to HVEM with improved affinity. In some embodiment, compared to wildtype LIGHT, the LIGHT muteins bind to DcR3 with reduced affinity. In some embodiment, compared to wildtype LIGHT, the LIGHT muteins bind to LTβR or HVEM with improved affinity and bind to DcR3 with reduced affinity.

In some embodiments, the LIGHT muteins maintain cross-reactivity across different species.

In some embodiments, the LTβR (e.g., human LTβR) binding LIGHT mutein includes an amino acid sequence set forth in any one of SEQ ID NOs: 1-75, 77-85 and SEQ ID NOs: 89-93.

In some embodiments, the LIGHT muteins bind to human LTβR with EC50 value no more than 1000 ng/ml, 800 ng/ml, 600 ng/ml, 400 ng/ml, 200 ng/ml, 100 ng/ml, 80 ng/ml, 70 ng/ml, 60 ng/ml or 50 ng/ml. In some embodiments, the LIGHT muteins bind to human LTβR with EC50 value no more than 2 nM, 1.8 nM, 1.5 nM, 1 nM, 0.9 nM, 0.8 nM, 0.7 nM, 0.6 nM, 0.5 nM or 0.4 nM.

In some embodiments, the LTβR (e.g., mouse LTβR) binding LIGHT mutein includes an amino acid sequence set forth in any one of SEQ ID NOs: 1-75, 77-85, and SEQ ID NOs: 89-93.

In some embodiments, the LIGHT muteins bind to mouse LTβR with EC50 value no more than 300 ng/ml, 200 ng/ml, 100 ng/ml, 80 ng/ml, 60 ng/ml, 50 ng/ml, 40 ng/ml or 30 ng/ml. In some embodiments, the LIGHT muteins bind to mouse LTβR with EC50 value no more than 3 nM, 2 nM, 1.5 nM, 1 nM, 0.9 nM, 0.8 nM, 0.7 nM, 0.6 nM, 0.5 nM or 0.4 nM.

In some embodiments, the HVEM (e.g., human HVEM) binding LIGHT mutein includes an amino acid sequence set forth in any one of SEQ ID NOs: 12-75, 77-85 and SEQ ID NOs: 89-93.

In some embodiments, the LIGHT muteins bind to human HVEM with EC50 value no more than 200 ng/ml, 150 ng/ml, 100 ng/ml, 80 ng/ml, 70 ng/ml, 60 ng/ml, 50 ng/ml or 40 ng/ml. In some embodiments, the LIGHT muteins bind to human HVEM with EC50 value no more than 10 nM, 8 nM, 7 nM, 6 nM, 3 nM, 2 nM, 1 nM, 0.8 nM, 0.7 nM, 0.6 nM, or 0.5 nM.

In some embodiments, the LIGHT muteins don't bind to human HVEM or substantially do not bind to human HVEM. In some embodiments, the HVEM (e.g., human HVEM) non-binding LIGHT mutein includes an amino acid sequence set forth in any one of SEQ ID NOs: 1-11.

In some embodiments, the HVEM (e.g., mouse HVEM) binding LIGHT mutein includes an amino acid sequence set forth in any one of SEQ ID NOs: 3-52.

In some embodiments, the HVEM (e.g., mouse HVEM) non-binding LIGHT mutein includes an amino acid sequence set forth in any one of SEQ ID NOs: 1-2.

In some embodiments, LIGHT mutein with reduced affinity to DcR3 (e.g., human DcR3) includes an amino acid sequence set forth in any one of SEQ ID NOs: 1-2, 52, 58, 61 and 89-93.

In some embodiments, LIGHT muteins don't bind to human DcR3 or substantially do not bind to human DcR3. In some embodiments, LIGHT muteins bind to human DcR3 with EC50 value more than 0.1 nM, 0.2 nM, 0.3 nM or 0.5 nM.

Improved affinity to LTβR or HVEM benefits for enhancing the efficacy of LIGHT muteins when preventing, treating or diagnosing a LIGHT-related disease, disorder or condition. Reduced affinity to DcR3 helps to minimize the toxicity caused by LIGHT-DcR3 interaction.

The LIGHT muteins provided herein optimize efficacy while minimizing potential toxicity.

### Isolated Polynucleotide

Encompassed within the present disclosure is an isolated polynucleotide encoding the LIGHT muteins described above. Aspects of the present disclosure include polynucleotide variants (e.g., due to degeneracy) that encode the amino acid sequences described herein.

Nucleotide sequences corresponding to the amino acid sequences described herein, can be obtained by “back-translation” from the amino acid sequences. The well-known polymerase chain reaction (PCR) procedure can be employed to isolate and amplify a DNA sequence encoding LIGHT muteins.

The isolated polynucleotide includes DNA and RNA in both single-stranded and double-stranded form, as well as the corresponding complementary sequences. An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)).

As will be appreciated by those skilled in the art, due to the degeneracy of the genetic code, each LIGHT mutein is encoded by an extremely large number of nucleic acids, each of which is within the scope of the disclosure and can be made using standard techniques. Thus, having identified a particular amino acid sequence, those skilled in the art could make any number of different nucleic acids, by simply modifying the sequence of one or more codons in a way that does not change the amino acid sequence of the encoded protein.

### Isolated Vectors

The disclosure also provides an isolated vector including the polynucleotide described above, the isolated vector acts as an expression system in the form of plasmids, a phagemid, a phage, a baculovirus, a cosmid or an artificial chromosome, transcription or expression cassettes which comprise at least one polynucleotide as above.

In some embodiments, the isolated vector also contain sequences for plasmid maintenance and for cloning and expression of exogenous nucleotide sequences, such as a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. The sequence for plasmid maintenance and for cloning and expression of exogenous nucleotide sequences could be obtained by any of several methods well known in the art.

The isolated vector constructed may be inserted into a suitable host cell for amplification and/or polypeptide expression. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, for example, in Sambrook et al., 2001, supra.

### Host Cells

A host cell containing the isolated vector provided herein may be eukaryotic or prokaryotic.

In some embodiments, the host cell is Mammalian cell line. Mammalian cell lines include, but are not limited to, immortalized cell lines available from the American Type Culture Collection (ATCC) and any cell lines used in an expression system known in the art can be used to make the LIGHT muteins of the disclosure.

Examples of suitable mammalian host cell lines include the COS-7 line of monkey kidney cells (ATCC CRL 1651) (Gluzman et al., 1981, Cell 23:175), Chinese hamster ovary (CHO) cells, or their derivatives such as Veggie CHO and related cell lines which grow in serum-free media (Rasmussen et al., 1998, Cytotechnology 28:31), HeLa cells, human embryonic kidney cells such as 293, 293 EBNA or MSR 293.

### Pharmaceutical Compositions

The disclosure provides a pharmaceutical composition comprising a therapeutically effective amount of LIGHT mutein together with a pharmaceutically acceptable carrier such as, a pharmaceutically effective diluents, carrier, solubilizer, emulsifier, preservative, and/or adjuvant. Pharmaceutical compositions of the disclosure include, but are not limited to, liquid, frozen, and lyophilized compositions.

Preferably, the pharmaceutically acceptable carrier, which acts as a formulation material are nontoxic to recipients at the dosages and concentrations employed. The optimal pharmaceutical composition will be determined by one skilled in the art depending upon, for example, the intended route of administration, delivery format and desired dosage.

In some embodiments, the pharmaceutical compositions can be selected for parenteral delivery. Preparation of such pharmaceutically acceptable compositions is within the skill of the art.

In some embodiments, the pharmaceutically acceptable carrier includes, but is not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); chelating agents (such as ethylenediamine tetraacetic acid (EDTA)); salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide). The pharmaceutically acceptable carrier improves effectiveness of the pharmaceutical composition and maximize the shelf-life of the pharmaceutical composition.

### Use of the LIGHT Mutein

The disclosure provides a use of the LIGHT mutein, the isolated polynucleotide, the isolated vector, the host cell or the pharmaceutical composition in the manufacture of a therapeutic agent (e.g., drug) for diagnosing, preventing, or treating a disease, disorder, or condition.

As use herein, the term “treat” of any disease refers to alleviating or ameliorating the disease, disorder, or condition (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent” of any disease refers to the prophylactic treatment of the disease; or delaying the onset or progression of the disease, disorder, or condition.

In another aspect, the disclosure provides a method of diagnosing, preventing or treating a disease, disorder, or condition in a subject in need thereof, including administrating to the subject a therapeutically effective amount of the LIGHT mutein, the isolated polynucleotide, the isolated vector, the host cell, or the pharmaceutical composition described above.

The therapeutically effective amount of the LIGHT mutein or the LIGHT mutein containing pharmaceutical composition, to be employed will depend, for example, upon the therapeutic context and objectives. One skilled in the art will appreciate that the appropriate dosage levels for treatment will vary depending, in part, upon the molecule delivered, the indication for which the LIGHT mutein is being used, the route of administration, and the size (body weight, body surface or organ size) and/or condition (the age and general health) of the patient. In certain embodiments, the clinician may titer the dosage and modify the route of administration to obtain the optimal therapeutic effect.

The subject refers to mammals, primates (e.g., humans, male or female), dogs, rabbits, guinea pigs, pigs, rats and mice. In certain embodiments, the subject is a primate. In yet other embodiments, the subject is human.

In some embodiments, the disease, disorder, or condition could be a LIGHT-related, such as, cancer. In some embodiments, at least one cell in tumors or tumor microenvironment expresses LIGHT receptors, e.g., LTβR, HVEM.

It will be readily apparent to those skilled in the art that other suitable modifications and adaptations of the present application described herein are obvious and may be made using suitable equivalents without departing from the scope of the disclosure or the embodiments disclosed herein.

### EXAMPLES

The following examples, both actual and prophetic, are provided for the purpose of illustrating specific embodiments or features of the present invention and are not intended to limit its scope.

### Example 1: Phage Library Design and Construction

The phagemid pComb3XSS (#VPT4013, Creative Biogene) was engineered to display human LIGHT (87-240) protein on the surfaces of M13 phage particles. The original sequence encoding TrxA of the pComb3XSS vector was replaced with the open reading frame encoding human LIGHT (87-240), a (G4S) 3 linker and the GCN4 peptide. The assembly of LIGHT as homotrimers was stabilized by the GCN4 peptide on the phage surface. The modified construct serves as a template for the library construction.

Three libraries, referred to as Library-1, Library-2 and Library-3, were constructed (Table-1). Library-1 was generated using the GeneMorph II Random Mutagenesis Kit (#200550, Agilent) following the manufacturer's protocol. The DNA fragment encoding human LIGHT (87-240) underwent three rounds of error-prone PCR using Mutazyme II DNA polymerase. The resulting PCR products were gel-extracted, purified and cloned into the phage vector. Library-2 and library-3 were constructed separately using mutagenic oligonucleotides, designed to introduce diversities at specific residues (Q117, G150, S160, T161, P171, E175, L220, D221, E222, L227) (Table-1). Degenerated primers with a mixture of bases (70-10-10-10) favoring wild-type sequences were synthesized (Genewiz, China). The resulting PCR products were inserted into the pComb3XSS phage display vector, and the reactions were transformed into the XL1-Blue cell (#DL1030, Weidi Bio, China).

E. coli XL1-blue cells harboring the phagemids were further infected with M13KO7 help phages (New England Biolabs, N0315SVIAL) and incubated overnight at 30° C. in the 2YT medium supplemented with 50 μg/ml ampicillin and 50 μg/ml kanamycin. Phages were precipitated from the culture medium using 20% PEG and 2.5M NaCl and subsequently resuspended in 0.5 ml of PBS supplemented with 20% glycerol. The concentration of phages was determined using the following formula: phage concentration=(OD269-OD320)×5×10¹² cfu/ml (cfu: colony forming unit).

TABLE 1
Phage library design.

| Library | Construction | Mutation sites of human |
|---|---|---|
| No. | method | LIGHT*(SEQ ID NO:87) |
| Library-1 | Error prone PCR | — |
| Library-2 | Soft randomization | Q117, P171, E175, L227 |
| Library-3 | Soft randomization | G150, S160, T161, L220, D221, E222 |

*Wherein the positions are defined with reference to SEQ ID NO: 87, and the position of the first amino acid of SEQ ID NO: 87 is defined as position 74.

### Example 2: Phage Panning and ELISA Analysis

Two panning strategies were implemented using phage libraries. A) The objective was to obtain mutants capable of binding to HVEM and LTβR. Sequential phage selection was performed using hHVEM, mHVEM, hLTβR and mLTβR as targets. B) The aim was to identify muteins that maintained LTβR binding while reducing HVEM binding. For the first three rounds of panning, hLTβR and mLTβR were used as targets. In the fourth round, the phages not bound to the plates coated with human- and mouse-HVEM were used in the subsequent positive selection using hLTβR and mLTβR.

For reducing DcR3 binding, the phage particles from the strategies A and B underwent an extra round of negative selection with plates coated with human DcR3 protein.

### Phage Panning

The HVEM and LTβR proteins, comprising the extracellular domain fused with an Fc fragment at the C-terminus, were obtained from SinoBiological (#10567-M03S), Novoprotein (#CX78) and Acrobiosystems (#HVM-H5258 and #LTR-H5251). The recombinant DcR3 protein was generated by linking human DcR3 residues 33-300 to the N-terminal of rabbit Fc. The recombinant proteins were expressed in expi293F cells (A14527, ThermoFisher) and affinity-purified using protein A resins, as previously described.

To screen desired mutants, 96-well immunoplates were coated overnight at 4° C. with antigens (5 μg/ml) listed in Table 2-1 and Table 2-2. The following morning, the plates were blocked with 2% BSA (bovine serum albumin, Sangon Biotech China) for 2 hours at room temperature. Phage solutions (1×10¹¹ phage) were added to the coated immunoplates and incubated for 1 hour at room temperature. The plates were washed five times with PBST (PBS, 0.5% Tween 20) and five times with PBS. Bound phages were eluted by adding 50 μl/well 100 mM glycine (pH 2.2) for 5 minutes. The eluant was transferred to a new tube and neutralized by adding 1/10 volume of 1M Tris buffer (pH 8.5). Eluted phages were amplified in E. coli XL1-Blue and used for further selection rounds.

TABLE 2-1
Phage panning strategy A and the target
proteins in the different rounds.

| Rounds | Target proteins in strategy A |
|---|---|
| First round | mouse LTβR |
| Second round | mouse HVEM |
| Third round | human LTβR |
| Fourth round | human HVEM |

TABLE 2-2
Phage panning strategy B and the target
proteins in the different rounds.

| Rounds | Target proteins in strategy B |
|---|---|
| First round | mouse LTβR |
| Second round | mouse LTβR |
| Third round | human LTβR |
| Fourth round | HVEM depletion + human LTβR |

### Phage ELISA

Individual clones from the fourth selection round were grown in 96-well deep well blocks in 100 μl of 2YT broth supplemented with 50 μg/ml ampicillin at 37° C. and 220 rpm for 5 hours. Then, 5 μl of helper phages were added to each well and incubated at 37° C. for 30 minutes. After centrifuging at 3,000 rpm for 10 minutes, the cells were resuspended and grown overnight in 150 μl/well of 2YT broth supplemented with 50 μg/ml ampicillin and 50 μg/ml kanamycin. The next day, the supernatant was collected by centrifugation at 3,000 rpm at 4° C. for 20 minutes and used for the ELISA assay to screen phage-displayed LIGHT muteins that bound to antigens but not BSA.

For the ELISA screening, 96-well ELISA plates were coated with target proteins (1 μg/ml in PBS) at 4° C. overnight and then blocked with 2% BSA for 2 hours at room temperature. The supernatant containing phage particles was added to the plates and incubated at room temperature for 1 hour. After incubation, the plates were washed five times with PBST. HRP (horseradish peroxidase) conjugated anti-M13 antibody (Sino Biological, China) was added to the wells and incubated at room temperature for 1 hour. Following another round of washing five times with PBST, 50 μl of TMB (3,3′, 5,5″-tetramethylbenzidine, #34029, ThemoFisher) was added to each well. After 5 minutes, the reaction was stopped by adding 50 μl stop solution (#C1058, Solarbio, China). The absorbance at 450 nM (OD450) was measured using a plate reader (SpectraMax M5, Molecule Devices). Wells coated with BSA were used as negative controls. Phage clones with an ELISA score ratio (target/BSA)>3 were considered positive clones.

### Example 3: Sequence Analysis of Positive Clones

Phagemids from the XL1-Blue cells that produced positive phage clones were extracted (BioSune, China) and sequenced. A total of 52 unique sequences were identified (SEQ ID NOs: 1-52, Table 3) with 1-5 amino acid mutations compared to the wild-type human LIGHT sequence (SEQ ID NO:87). Among these sequences, 11 mutants were derived from the HVEM-depletion group, and they were found not to bind HVEM protein. The remaining mutants exhibited cross-reactivity to human and mouse HVEM and LTβR receptors (Table 3).

### Example 4: NGS Sequencing and LIGHT Mutant Design by Machine Learning

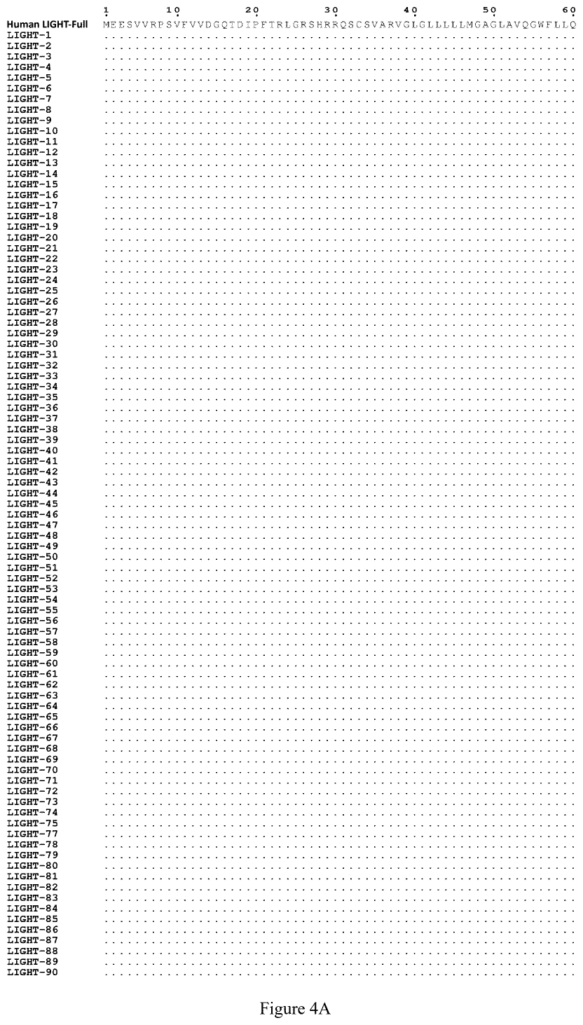

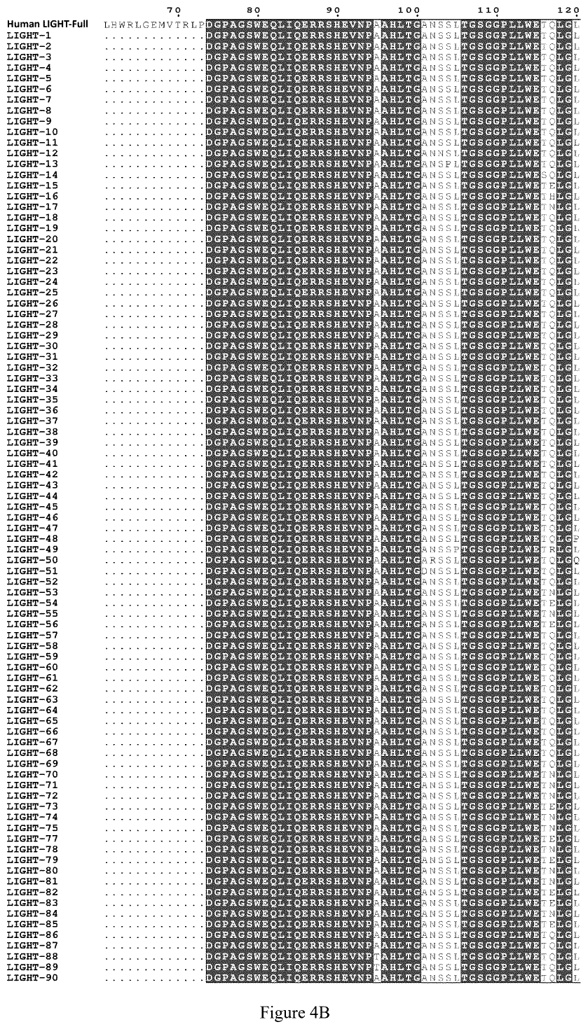

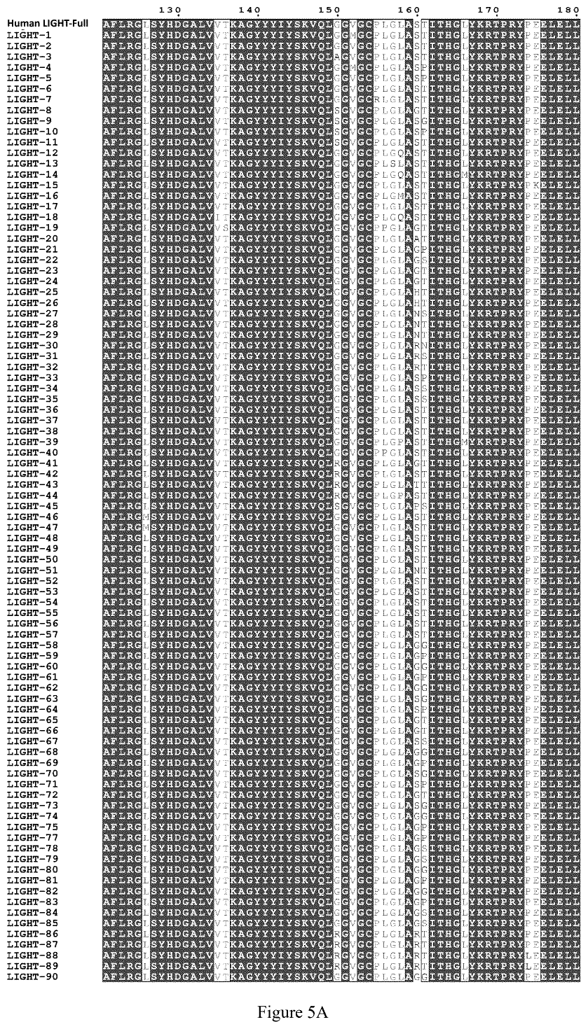

For further affinity improvement through deep sequencing, phagemids were isolated from the phages capable of binding target proteins, as well as those that exhibited nonspecific binding to BSA. The segment of the LIGHT protein was then PCR amplified and purified (DC301, Vazyme, China). Amplicons were prepared using the VATHS Universal DNA Library Prep Kit (Vazyme #ND607-01), following the standard library preparation protocol. Adapter-ligated libraries underwent a single cycle of PCR and were subsequently sequenced on the Illumina Miseq system using paired-end 300-bp reads to cover the entire length of the amplicons. The sequencing results were used to predict high-affinity clones to the receptors by comparing the frequency of a given mutation at a specific position in the enriched samples against negative controls. A total of 33 LIGHT mutants containing 3-7 mutation sites (Table 3) (SEQ ID NOs: 53-85) were expressed and further characterized. The amino acid mutations and positions of all LIGHT muteins (SEQ IN NOs: 1-85 and 89-93) are summarized in Table 4 (see FIGS. 4 and 5). LIGHT muteins, LIGHT86-89 (SEQ IN NOs: 89-93), were identified through DcR3 depletion phage panning.

TABLE 3
LIGHT muteins from phage panning and machine learning

| | SEQ | PHAGE | | |
|---|---|---|---|---|
| | ID | ELISA | AMINO ACID SEQUENCE (single-letter code) | |
| NAME | NO. | RESULTS | The mutated amino acids of LIGHT muteins are in bond and italic. | |
| LIGHT | 86 | N.A | MEESVVRPSVFVVDGQTDIPFTRLGRSHRRQSCSVARVGLGLLLLLMGA | |
| (1-240) | | | GLAVQGWFLLQLHWRLGEMVTRLPDGPAGSWEQLIQERRSHEVNPAAH | |
| | | | LTGANSSLTGSGGPLLWETQLGLAFLRGLSYHDGALVVTKAGYYYIYSK | |
| | | | VQLGGVGCPLGLASTITHGLYKRTPRYPEELELLVSQQSPCGRATSSSRV | |
| | | | WWDSSFLGGVVHLEAGEKVVVRVLDERLVRLRDGTRSYFGAFMV | |
| LIGHT | 87 | mLTβR+/−, | DGPAGSWEQLIQERRSHEVNPAAHLTGANSSLTGSGGPLLWETQLGLAF | |
| (74- | | hLTβR+/−, | LRGLSYHDGALVVTKAGYYYIYSKVQLGGVGCPLGLASTITHGLYKRTP | |
| 240) | | mHVEM+/−, | RYPEELELLVSQQSPCGRATSSSRVWWDSSFLGGVVHLEAGEKVVVRVL | |
| | | hHVEM+/− | DERLVRLRDGTRSYFGAFMV | |
| LIGHT | 88 | N.A, | RRSHEVNPAAHLTGANSSLTGSGGPLLWETQLGLAFLRGLSYHDGALVV | |
| (87- | | | TKAGYYYIYSKVQLGGVGCPLGLASTITHGLYKRTPRYPEELELLVSQQS | |
| 240) | | | PCGRATSSSRVWWDSSFLGGVVHLEAGEKVVVRVLDERLVRLRDGTRS | |
| | | | YFGAFMV | |
| LIGHT- | 1 | mLTβR+, | DGPAGSWEQLIQERRSHEVNPAAHLTGANSSLTGSGGPLLWETQLGLAF | V152M |
| 1 | | hLTβR+, | LRGLSYHDGALVVTKAGYYYIYSKVQLGGMGCPLGLASTITHGLYKRTP | W198Q |
| | | mHVEM-, | RYPEELELLVSQQSPCGRATSSSRVWQDSSFLGGVVHLEAGEKVVVRVL | R228L |
| | | hHVEM− | DERLVRLLDGTRSYFGAFMV | |
| LIGHT- | 2 | mLTβR+, | DGPAGSWEQLIQERRSHEVNPAAHLTGANSSLTGSGGPLLWETQLGLAF | R189S |
| 2 | | hLTβR+, | LRGLSYHDGALVVTKAGYYYIYSKVQLGGVGCPLGLASTITHGLYKRTP | W198Q |
| | | mHVEM-, | RYPEELELLVSQQSPCGSATSSSRVWQDSSFLGGVVHLEAGEKVVVRVL | R228L |
| | | hHVEM− | DERLVRLLDGTRSYFGAFMV | |
| LIGHT- | 3 | mLTβR+, | DGPAGSWEQLIQERRSHEVNPAAHLTGANSSLTGSGGPLLWETQLGLAF | G150A |
| 3 | | hLTβR+, | LRGLSYHDGALVVTKAGYYYIYSKVQLAVGCPLGLASTITHGLYKRTP | L220S |
| | | mHVEM+, | RYPEELELLVSQQSPCGRATSSSRVWWDSSFLGGVVHLEAGEKVVVRVS | |
| | | hHVEM− | DERLVRLRDGTRSYFGAFMV | |
| LIGHT- | 4 | mLTβR+, | DGPAGSWEQLIQERRSHEVNPAAHLTGANSSLTGSGGPLLWETQLGLAF | V152M |
| 4 | | hLTβR+, | LRGLSYHDGALVVTKAGYYYIYSKVQLGGMGCPLGLASPITHGLYKRTP | T161P |
| | | mHVEM+, | RYPEELELLVSQQSPCGRATSSSRVWWDSSFLGGVVHLEAGEKVVVRVL | R228L |
| | | hHVEM− | DERLVRLLDGTRSYFGAFMV | |
| LIGHT- | 5 | mLTβR+, | DGPAGSWEQLIQERRSHEVNPAAHLTGANSSLTGSGGPLLWETQLGLAF | T161P |
| 5 | | hLTβR+, | LRGLSYHDGALVVTKAGYYYIYSKVQLGGVGCPLGLASPITHGLYKRTP | R189S |
| | | mHVEM+, | RYPEELELLVSQQSPCGSATSSSRVWQDSSFLGGVVHLEAGEKVVVRVL | W198Q |
| | | hHVEM− | DERLVRLLDGTRSYFGAFMV | R228L |
| LIGHT- | 6 | mLTβR+, | DGPAGSWEQLIQERRSHEVNPAAHLTGANSSLTGSGGPLLWETQLGLAF | W198Q |
| 6 | | hLTβR+, | LRGLSYHDGALVVTKAGYYYIYSKVQLGGVGCPLGLASTITHGLYKRTP | L220N |
| | | mHVEM+, | RYPEELELLVSQQSPCGRATSSSRVWQDSSFLGGVVHLEAGEKVVVRVN | D221G |
| | | hHVEM− | GERLVRLRDGTRSYFGAFMV | |
| LIGHT- | 7 | mLTβR+, | DGPAGSWEQLIQERRSHEVNPAAHLTGANSSLTGSGGPLLWETQLGLAF | P155R |
| 7 | | hLTβR+, | LRGLSYHDGALVVTKAGYYYIYSKVQLGGVGCRLGLASTITHGLYKRTP | L220Q |
| | | mHVEM+, | RYPEELELLVSQQSPCGRATSSSRVWWDSSFLGGVVHLEAGEKVVVRV | R232H |
| | | hHVEM− | QDERLVRLRDGTHSYFGAFMV | |
| LIGHT- | 8 | mLTβR+, | DGPAGSWEQLIQERRSHEVNPAAHLTGANSSLTGSGGPLLWETQLGLAF | G150S |
| 8 | | hLTβR+, | LRGLSYHDGALVVTKAGYYYIYSKVQLSGVGCPLGLAGTITHGLYKRTP | S160G |
| | | mHVEM+, | RYPEELELLVSQQSPCGRATSSSRVWWDSSFLGGVVHLEAGEKVVVRVS | L220S |
| | | hHVEM− | DERLVRLRDGTRSYFGAFMV | |
| LIGHT- | 9 | mLTβR+, | DGPAGSWEQLIQERRSHEVNPAAHLTGANSSLTGSGGPLLWETQLGLAF | G150S |
| 9 | | hLTβR+, | LRGLSYHDGALVVTKAGYYYIYSKVQLSGVGCPLGLASGITHGLYKRTP | T161G |
| | | mHVEM+, | RYPEELELLVSQQSPCGRATSSSRVWWDSSFLGGVVHLEAGEKVVVRVS | L220S |
| | | hHVEM− | DERLVRLRDGTRSYFGAFMV | |
| LIGHT- | 10 | mLTβR+, | DGPAGSWEQLIQERRSHEVNPAAHLTGANSSLTGSGGPLLWETQLGLAF | G150S |
| 10 | | hLTβR+, | LRGLSYHDGALVVTKAGYYYIYSKVQLSGVGCPLGLASPITHGLYKRTP | T161P |
| | | mHVEM+, | RYPEELELLVSQQSPCGRATSSSRVWWDSSFLGGVVHLEAGEKVVVRVS | L220S |
| | | hHVEM− | DERLVRLRDGTRSYFGAFMV | |
| LIGHT- | 11 | mLTβR+, | DGPAGSWEQLIQERRSHEVNPAAHLTGANSSLTGSGGPLLWETQLGLAF | G150S |
| 11 | | hLTβR+, | LRGLSYHDGALVVTKAGYYYIYSKVQLSGVGCPLGLASTITHGLYKRTP | L220S |
| | | mHVEM+, | RYPEELELLVSQQSPCGRATSSSRVWWDSSFLGGVVHLEAGEKVVVRVS | |
| | | hHVEM− | DERLVRLRDGTRSYFGAFMV | |
| LIGHT- | 12 | mLTβR+, | DGPAGSWEQLIQERRSHEVNPAAHLTGANNSLTGSGGPLLWETQLGLAF | L158Q |
| 12 | | hLTβR+, | LRGLSYHDGALVVTKAGYYYIYSKVQLGGVGCPLGQASTITHGLYKRTP | K214E |
| | | mHVEM+, | RYPEELELLVSQQSPCGRATSSSRVWWDSSFLGGVVHLEAGEEVVVRVL | |
| | | hHVEM+ | DERLVRLRDGTRSYFGAFMV | |
| LIGHT- | 13 | mLTβR+, | DGPAGSWEQLIQERRSHEVNPAAHLTGANSPLTGSGGPLLWETQLGLAF | S104P |
| 13 | | hLTβR+, | LRGLSYHDGALVVTKAGYYYIYSKVQLGGVGCPLSLASTITHGLYKRTP | G157S |
| | | mHVEM+, | RYPEELELLVSQQSPCGRATSSSRVWWDSSFLGGVVYLEAGEKVVVRVR | H208Y |
| | | hHVEM+ | DERLVRLRDGTRSYFGAFMV | L220R |
| LIGHT- | 14 | mLTβR+, | DGPAGSWEQLIQERRSHEVNPAAHLTGANSSLTGSGGPLLWESQLGLAF | L158Q |
| 14 | | hLTβR+, | LRGLSYHDGALVVTKAGYYYIYSKVQLGGVGCPLGQASTITHGMYKRT | L166M |
| | | mHVEM+, | PRYPEELELLVSQQSPCGRATSSSRVWWDSSFLGGVVHLEAGEKVVVRV | |
| | | hHVEM+ | LDERLVRLRDGTRSYFGAFMV | |
| LIGHT- | 15 | mLTβR+, | DGPAGSWEQLIQERRSHEVNPAAHLTGANSSLTGSGGPLLWETELGLAF | Q117E |
| 15 | | hLTβR+, | LRGLSYHDGALVVTKAGYYYIYSKVQLGGVGCPLGLASTITHGLYKRTP | E175K |
| | | mHVEM+, | RYPKELELLVSQQSPCGRATSSSRVWWDSSFLGGVVHLEAGEEVVVRVL | K214E |
| | | hHVEM+ | DERLVRTRDGTRSYFGAFMV | L227T |
| LIGHT- | 16 | mLTβR+, | DGPAGSWEQLIQERRSHEVNPAAHLTGANSSLTGSGGPLLWETHLGLAF | Q117H |
| 16 | | hLTβR+, | LRGLSYHDGALVVTKAGYYYIYSKVQLGGVGCPLGMASTITHGLYKRT | L158M |
| | | mHVEM+, | PRYPEELELLVSQQSPCGRATSSSRVWWDSSFLGGVVHLEAGDKVVVRV | E213D |
| | | hHVEM+ | LDERLVRLRDGTRSYFGAFMV | |
| LIGHT- | 17 | mLTβR+, | DGPAGSWEQLIQERRSHEVNPAAHLTGANSSLTGSGGPLLWETNLGLAF | H208Y |
| 17 | | hLTβR+, | LRGLSYHDGALVVTKAGYYYIYSKVQLGGVGCPLGLASTITHGLYKRTP | Q117N |
| | | mHVEM+, | RYPEELELLVSQQSPCGRATSSSRVWWDSSFLGGVVYLEAGEKVVVRVL | |
| | | hHVEM+ | DERLVRLRDGTRSYFGAFMV | |
| LIGHT- | 18 | mLTβR+, | DGPAGSWEQLIQERRSHEVNPAAHLTGANSSLTGSGGPLLWETQLGLAF | L158Q |
| 18 | | hLTβR+, | LRGLSYHDGALVITKAGYYYIYSKVQLGGVGCPLGQASTITHGLYKRTP | K214E |
| | | mHVEM+, | RYPEELELLVSQQSPCGRATSSSRVWWDSSFLGGVVHLEAGEEVVVRVL | E222K |
| | | hHVEM+ | DKRLVRLRDGTRSYFGAFMV | |
| LIGHT- | 19 | mLTβR+, | DGPAGSWEQLIQERRSHEVNPAAHLTGANSSLTGSGGPLLWETQLGLAF | L156P |
| 19 | | hLTβR+, | LRGLSYHDGALVVSKAGYYYIYSKVQLGGVGCPPGLAGTITHGLYKRTP | S160G |
| | | mHVEM+, | RYPEELELLVSQQSPCGRATSSSRVWWDSSFLGGVVHLEAGEKVVVRV | L220M |
| | | hHVEM+ | MDERLVRLRDGTRSYFGAFMV | |
| LIGHT- | 20 | mLTβR+, | DGPAGSWEQLIQERRSHEVNPAAHLTGANSSLTGSGGPLLWETQLGLAF | S160A |
| 20 | | hLTβR+, | LRGLSYHDGALVVTKAGYYYIYSKVQLGGVGCPLGLAATITHGLYKRTP | L220S |
| | | mHVEM+, | RYPEELELLVSQQSPCGRATSSSRVWWDSSFLGGVVHLEAGEKVVVRVS | E222D |
| | | hHVEM+ | DDRLVRLRDGTRSYFGAFMV | |
| LIGHT- | 21 | mLTβR+, | DGPAGSWEQLIQERRSHEVNPAAHLTGANSSLTGSGGPLLWETQLGLAF | S160G |
| 21 | | hLTβR+, | LRGLSYHDGALVVTKAGYYYIYSKVQLGGVGCPLGLAGPITHGLYKRTP | T161P |
| | | mHVEM+, | RYPEELELLVSQQSPCGRATSSSRVWWDSSFLGGVVHLEAGEKVVVRVT | L220T |
| | | hHVEM+ | DERLVRLRDGTRSYFGAFMV | |
| LIGHT- | 22 | mLTβR+, | DGPAGSWEQLIQERRSHEVNPAAHLTGANSSLTGSGGPLLWETQLGLAF | S160G |
| 22 | | hLTβR+, | LRGLSYHDGALVVTKAGYYYIYSKVQLGGVGCPLGLAGSITHGLYKRTP | T161S |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 23 | mLTβR+, | DGPAGSWEQLIQERRSHEVNPAAHLTGANSSLTGSGGPLLWETQLGLAF | S160G |
| 23 | | hLTβR+, | LRGLSYHDGALVVTKAGYYYIYSKVQLGGVGCPLGLAGTITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 24 | mLTβR+, | DGPAGSWEQLIQERRSHEVNPAAHLTGANSSLTGSGGPLLWETQLGLAF | S160G |
| 24 | | hLTβR+, | LRGLSYHDGALVVTKAGYYYIYSKVQLGGVGCPLGLAGTITHGLYKRTP | A190T |
| | | mHVEM+, | RYPEELELLVSQQSPCGRTTSSSRVWWDSSFLGGVVHLEAGEKVVVRVS | L220S |
| | | hHVEM+ | DERLVRLRDGTRSYFGAFMV | |
| LIGHT- | 25 | mLTβR+, | DGPAGSWEQLIQERRSHEVNPAAHLTGANSSLTGSGGPLLWETQLGLAF | S160H |
| 25 | | hLTβR+, | LRGLSYHDGALVVTKAGYYYIYSKVQLGGVGCPLGLAHTITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 26 | mLTβR+, | DGPAGSWEQLIQERRSHEVNPAAHLTGANSSLTGSGGPLLWETQLGLAF | S160H |
| 26 | | hLTβR+, | LRGLSYHDGALVVTKAGYYYIYSKVQLGGVGCPLGLAHTITHGLYKRTP | L220S |
| | | mHVEM+, | RYPEELELLVSQQSPCGRATSSSRVWWDSSFLGGVVHLEAGEKVVVRVS | E222Q |
| | | hHVEM+ | DQRLVRLRDGTRSYFGAFMV | |
| LIGHT- | 27 | mLTβR+, | DGPAGSWEQLIQERRSHEVNPAAHLTGANSSLTGSGGPLLWETQLGLAF | S160N |
| 27 | | hLTβR+, | LRGLSYHDGALVVTKAGYYYIYSKVQLGGVGCPLGLANSITHGLYKRTP | T161S |
| | | mHVEM+, | RYPEELELLVSQQSPCGRATSSSRVWWDSSFLGGVVHLEAGEKVVVRVS | L220S |
| | | hHVEM+ | DDRLVRLRDGTRSYFGAFMV | E222D |
| LIGHT- | 28 | mLTβR+, | DGPAGSWEQLIQERRSHEVNPAAHLTGANSSLTGSGGPLLWETQLGLAF | S160N |
| 28 | | hLTβR+, | LRGLSYHDGALVVTKAGYYYIYSKVQLGGVGCPLGLANTITHGLYKRTP | K214E |
| | | mHVEM+, | RYPEELELLVSQQSPCGRATSSSRVWWDSSFLGGVVHLEAGEEVVVRVL | L227M |
| | | hHVEM+ | DERLVRMRDGTRSYFGAFMV | |
| LIGHT- | 29 | mLTβR+, | DGPAGSWEQLIQERRSHEVNPAAHLTGANSSLTGSGGPLLWETQLGLAF | S160N |
| 29 | | hLTβR+, | LRGLSYHDGALVVTKAGYYYIYSKVQLGGVGCPLGLANTITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 30 | mLTβR+, | DGPAGSWEQLIQERRSHEVNPAAHLTGANSSLTGSGGPLLWETQLGLAF | S160R |
| 30 | | hLTβR+, | LRGLSYHDGALVVTKAGYYYIYSKVQLGGVGCPLGLARNITHGLYKRTP | T161N |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 31 | mLTβR+, | DGPAGSWEQLIQERRSHEVNPAAHLTGANSSLTGSGGPLLWETQLGLAF | S160R |
| 31 | | hLTβR+, | LRGLSYHDGALVVTKAGYYYIYSKVQLGGVGCPLGLARSITHGLYKRTP | T161S |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 32 | mLTβR+, | DGPAGSWEQLIQERRSHEVNPAAHLTGANSSLTGSGGPLLWETQLGLAF | S160R |
| 32 | | hLTβR+, | LRGLSYHDGALVVTKAGYYYIYSKVQLGGVGCPLGLARTITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 33 | mLTβR+, | DGPAGSWEQLIQERRSHEVNPAAHLTGANSSLTGSGGPLLWETQLGLAF | T161P |
| 33 | | hLTβR+, | LRGLSYHDGALVVTKAGYYYIYSKVQLGGVGCPLGLASPITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 34 | mLTβR+, | DGPAGSWEQLIQERRSHEVNPAAHLTGANSSLTGSGGPLLWETQLGLAF | T161S |
| 34 | | hLTβR+, | LRGLSYHDGALVVTKAGYYYIYSKVQLGGVGCPLGLASSITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 35 | mLTβR+, | DGPAGSWEQLIQERRSHEVNPAAHLTGANSSLTGSGGPLLWETQLGLAF | T161S |
| 35 | | hLTβR+, | LRGLSYHDGALVVTKAGYYYIYSKVQLGGVGCPLGLASSITHGLYKRTP | L220S |
| | | mHVEM+, | RYPEELELLVSQQSPCGRATSSSRVWWDSSFLGGVVHLEAGEKVVVRVS | E222N |
| | | hHVEM+ | DNRLVRLRDGTRSYFGAFMV | |
| LIGHT- | 36 | mLTβR+, | DGPAGSWEQLIQERRSHEVNPAAHLTGANSSLTGSGGPLLWETQLGLAF | H208Y |
| 36 | | hLTβR+, | LRGLSYHDGALVVTKAGYYYIYSKVQLGGVGCPLGLASTITHGLYKRTP | |
| | | mHVEM+, | RYPEELELLVSQQSPCGRATSSSRVWWDSSFLGGVVYLEAGEKVVVRVL | |
| | | hHVEM+ | DERLVRLRDGTRSYFGAFMV | |
| LIGHT- | 37 | mLTβR+, | DGPAGSWEQLIQERRSHEVNPAAHLTGANSSLTGSGGPLLWETQLGLAF | A190T |
| 37 | | hLTβR+, | LRGLSYHDGALVVTKAGYYYIYSKVQLGGVGCPLGLASTITHGLYKRTP | F202Y |
| | | mHVEM+, | RYPEELELLVSQQSPCGRTTSSSRVWWDSSYLGGVVHLEAGEEVVVRVL | K214E |
| | | hHVEM+ | DERLVRLRDGTRSYFGAFMV | |
| LIGHT- | 38 | mLTβR+, | DGPAGSWEQLIQERRSHEVNPAAHLTGANSSLTGSGGPLLWETQLGLAF | Q184R |
| 38 | | hLTβR+, | LRGLSYHDGALVVTKAGYYYIYSKVQLGGVGCPLGLASTITHGLYKRTP | W198Q |
| | | mHVEM+, | RYPEELELLVSQRSPCGRATSSSRVWQDSSFLGGVVHLEAGEKVVVRVL | |
| | | hHVEM+ | DERLVRLRDGTRSYFGAFMV | |
| LIGHT- | 39 | mLTβR+, | DGPAGSWEQLIQERRSHEVNPAAHLTGANSSLTGSGGPLLWETQLGLAF | L158P |
| 39 | | hLTβR+, | LRGLSYHDGALVVTKAGYYYIYSKVQLGGVGCPLGPASTITHGMYKRT | L166M |
| | | mHVEM+, | PRYPEELELLVSQQSPCGRATSSSRVWWDSSFLGGVVHLEAGEKVVVRV | L220M |
| | | hHVEM+ | MDERLVRLRDGTRSYFGAFMV | |
| LIGHT- | 40 | mLTβR+, | DGPAGSWEQLIQERRSHEVNPAAHLTGANSSLTGSGGPLLWETQLGLAF | L156P |
| 40 | | hLTβR+, | LRGLSYHDGALVVTKAGYYYIYSKVQLGGVGCPPGLASTITHGLYKRTP | H208Y |
| | | mHVEM+, | RYPEELELLVSQQSPCGRATSSSRVWWDSSFLGGVVYLEAGEKVVVRVL | |
| | | hHVEM+ | DERLVRLRDGTRSYFGAFMV | |
| LIGHT- | 41 | mLTβR+, | DGPAGSWEQLIQERRSHEVNPAAHLTGANSSLTGSGGPLLWETQLGLAF | G150R |
| 41 | | hLTβR+, | LRGLSYHDGALVVTKAGYYYIYSKVQLRGVGCPLGLAGTITHGLYKRTP | S160G |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 42 | mLTβR+, | DGPAGSWEQLIQERRSHEVNPAAHLTGANSSLTGSGGPLLWETQLGLAF | G150R |
| 42 | | hLTβR+, | LRGLSYHDGALVVTKAGYYYIYSKVQLRGVGCPLGLASTITHGLYKRTP | L220S |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 43 | mLTβR+, | DGPAGSWEQLIQERRSHEVNPAAHLTGANSSLTGSGGPLLWETQLGLAF | G150R |
| 43 | | hLTβR+, | LRGLSYHDGALVVTKAGYYYIYSKVQLRGVGCPLGLATTITHGLYKRTP | S160T |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 44 | mLTβR+, | DGPAGSWEQLIQERRSHEVNPAAHLTGANSSLTGSGGPLLWETQLGLAF | G150R |
| 44 | | hLTβR+, | LRGLSYHDGALVVTKAGYYYIYSKVQLRGVGCPLGPASTITHGLYKRTP | L158P |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 45 | mLTβR+, | DGPAGSWEQLIQERRSHEVNPAAHLTGANSSLTGSGGPLLWETQLGLAF | G150S |
| 45 | | hLTβR+, | LRGLSYHDGALVVTKAGYYYIYSKVQLSGVGCPLGLAPSITHGLYKRTP | S160P |
| | | mHVEM+, | RYPEELELLVSQQSPCGRATSSSRVWWDSSFLGGVVHLEAGEKVVVRVS | T161S |
| | | hHVEM+ | DEHLVRLRDGTRSYFGAFMV | L220S |
| | | | | R223H |
| LIGHT- | 46 | mLTβR+, | DGPAGSWEQLIQERRSHEVNPAAHLTGANSSLTGSGGPLLWETQLGLAF | L126M |
| 46 | | hLTβR+, | LRGMSYHDGALVVTKAGYYYIYSKVQLGGVGCPLGLASTITHGLYKRT | K214E |
| | | mHVEM+, | PRYPEELELLVSQQSPCGRATSSSRVWWDSSFLGGVVHLEAGEEVVVRV | |
| | | hHVEM+ | LDERLVRLRDGTRSYFGAFMV | |
| LIGHT- | 47 | mLTβR+, | DGPAGSWEQLIQERRSHEVNPAAHLTGANSSLTGSGGPLLWETQLGLAF | L126M |
| 47 | | hLTβR+, | LRGMSYHDGALVVTKAGYYYIYSKVQLGGVGCPLGLASTITHGLYKRT | A190V |
| | | mHVEM+, | PRYPEELELLVSQQSPCGRVTSSSRVWWDSSFLGGVVYLEAGEKVVVRV | H208Y |
| | | hHVEM+ | LDERLVRLRDGTRSYFGAFMV | |
| LIGHT- | 48 | mLTβR+, | DGPAGSWEQLIQERRSHEVNPAAHLTGANSSLTGSGGPLLWETQLGPAF | L120P |
| 48 | | hLTβR+, | LRGLSYHDGALVVTKAGYYYIYSKVQLGGVGCPLGLASTITHGLYKRTP | L220R |
| | | mHVEM+, | RYPEELELLVSQQSPCGRATSSSRVWWDSSFLGGVVHLEAGEKVVVRVR | |
| | | hHVEM+ | DERLVRLRDGTRSYFGAFMV | |
| LIGHT- | 49 | mLTβR+, | DGPAGSWEQLIQERRSHEVNPAAHLTGANSSPTGSGGPLLWETRLGLAF | L105P |
| 49 | | hLTβR+, | LRGLSYHDGALVVTKAGYYYIYSKVQLGGVGCPLGLASTITHGLYKRTP | Q117R |
| | | mHVEM+, | RYPEELELLVSQQSPCGRATSSSRVWWDSSFLGGVVHLEAGEKVVVRV | L220Q |
| | | hHVEM+ | QDERLVRLRDGTRSYFGAFMV | |
| LIGHT- | 50 | mLTβR+, | DGPAGSWEQLIQERRSHEVNPAAHLTGARSSLTGSGGPLLWETQLGQAF | N102R |
| 50 | | hLTβR+, | LRGLSYHDGALVVTKAGYYYIYSKVQLGGVGCPLGLASTITHGLYKRTP | L120Q |
| | | mHVEM+, | RYPEELELLVSQQSPCGRATSSSRVWWDSSFLGGVVRLEAGEEVVVRVL | H208R |
| | | hHVEM+ | DERLVRLRDGTRSYFGAFMV | K214E |
| LIGHT- | 51 | mLTβR+, | DGPAGSWEQLIQERRSHEVNPAAHLTGDNSSLTGSGGPLLWETQLGLAF | A101D |
| 51 | | hLTβR+, | LRGLSYHDGALVVTKAGYYYIYSKVQLGGVGCPLGLANTITHGLYKRTP | S160N |
| | | mHVEM+, | RYPEELELLVSQQSPCGRATSSSRVWWDSSFLGGVVHLEAGEKVVVRVS | L220S |
| | | hHVEM+ | DERLVRLRDGTRSYFGAFMV | |
| LIGHT- | 52 | mLTβR+, | DGPAGSWEQLIQERRSHEVNPAAHLTGANSSLTGSGGPLLWETQLGLAF | L220S |
| 52 | | hLTβR+, | LRGLSYHDGALVVTKAGYYYIYSKVQLGGVGCPLGLASTITHGLYKRTP | |
| | | mHVEM+, | RYPEELELLVSQQSPCGRATSSSRVWWDSSFLGGVVHLEAGEKVVVRVS | |
| | | hHVEM+ | DERLVRLRDGTRSYFGAFMV | |
| LIGHT- | 53 | mLTβR+, | DGPAGSWEQLIQERRSHEVNPAAHLTGANSSLTGSGGPLLWETNLGLAF | Q117N |
| 53 | | hLTβR+, | LRGLSYHDGALVVTKAGYYYIYSKVQLGGVGCPLGLASTITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 54 | mLTβR+, | DGPAGSWEQLIQERRSHEVNPAAHLTGANSSLTGSGGPLLWETELGLAF | Q117E |
| 54 | | hLTβR+, | LRGLSYHDGALVVTKAGYYYIYSKVQLGGVGCPLGLASTITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 55 | mLTβR+, | DGPAGSWEQLIQERRSHEVNPAAHLTGANSSLTGSGGPLLWETNLGLAF | Q117N |
| 55 | | hLTβR+, | LRGLSYHDGALVVTKAGYYYIYSKVQLGGVGCPLGLASTITHGLYKRTP | L220T |
| | | hHVEM+ | RYPEELELLVSQQSPCGRATSSSRVWWDSSFLGGVVHLEAGEKVVVRVT | L227T |
| | | | DERLVRTRDGTRSYFGAFMV | |
| LIGHT- | 56 | mLTβR+, | DGPAGSWEQLIQERRSHEVNPAAHLTGANSSLTGSGGPLLWETELGLAF | Q117E |
| 56 | | hLTβR+, | LRGLSYHDGALVVTKAGYYYIYSKVQLGGVGCPLGLASTITHGLYKRTP | L220T |
| | | hHVEM+ | RYPEELELLVSQQSPCGRATSSSRVWWDSSFLGGVVHLEAGEKVVVRVT | L227T |
| | | | DERLVRTRDGTRSYFGAFMV | |
| LIGHT- | 57 | mLTβR+, | DGPAGSWEQLIQERRSHEVNPAAHLTGANSSLTGSGGPLLWETQLGLAF | L220S |
| 57 | | hLTβR+, | LRGLSYHDGALVVTKAGYYYIYSKVQLGGVGCPLGLASTITHGLYKRTP | L227T |
| | | hHVEM+ | RYPEELELLVSQQSPCGRATSSSRVWWDSSFLGGVVHLEAGEKVVVRVS | |
| | | | DERLVRTRDGTRSYFGAFMV | |
| LIGHT- | 58 | mLTβR+, | DGPAGSWEQLIQERRSHEVNPAAHLTGANSSLTGSGGPLLWETQLGLAF | S160G |
| 58 | | hLTβR+, | LRGLSYHDGALVVTKAGYYYIYSKVQLGGVGCPLGLAGGITHGLYKRT | T161G |
| | | hHVEM+ | PRYPEELELLVSQQSPCGRATSSSRVWWDSSFLGGVVHLEAGEKVVVRV | L220S |
| | | | SDERLVRLRDGTRSYFGAFMV | |
| LIGHT- | 59 | mLTβR+, | DGPAGSWEQLIQERRSHEVNPAAHLTGANSSLTGSGGPLLWETQLGLAF | S160G |
| 59 | | hLTβR+, | LRGLSYHDGALVVTKAGYYYIYSKVQLGGVGCPLGLAGPITHGLYKRTP | T161P |
| | | hHVEM+ | RYPEELELLVSQQSPCGRATSSSRVWWDSSFLGGVVHLEAGEKVVVRVS | L220S |
| | | | DERLVRLRDGTRSYFGAFMV | |
| LIGHT- | 60 | mLTβR+, | DGPAGSWEQLIQERRSHEVNPAAHLTGANSSLTGSGGPLLWETQLGLAF | S160G |
| 60 | | hLTβR+, | LRGLSYHDGALVVTKAGYYYIYSKVQLGGVGCPLGLAGGITHGLYKRT | T161G |
| | | hHVEM+ | PRYPEELELLVSQQSPCGRATSSSRVWWDSSFLGGVVHLEAGEKVVVRV | L220S |
| | | | SDSRLVRLRDGTRSYFGAFMV | E222S |
| LIGHT- | 61 | mLTβR+, | DGPAGSWEQLIQERRSHEVNPAAHLTGANSSLTGSGGPLLWETQLGLAF | S160G |
| 61 | | hLTβR+, | LRGLSYHDGALVVTKAGYYYIYSKVQLGGVGCPLGLAGPITHGLYKRTP | T161P |
| | | hHVEM+ | RYPEELELLVSQQSPCGRATSSSRVWWDSSFLGGVVHLEAGEKVVVRVS | L220S |
| | | | DSRLVRLRDGTRSYFGAFMV | E222S |
| LIGHT- | 62 | mLTβR+, | DGPAGSWEQLIQERRSHEVNPAAHLTGANSSLTGSGGPLLWETQLGLAF | S160G |
| 62 | | hLTβR+, | LRGLSYHDGALVVTKAGYYYIYSKVQLGGVGCPLGLAGGITHGLYKRT | T161G |
| | | hHVEM+ | PRYPEELELLVSQQSPCGRATSSSRVWWDSSFLGGVVHLEAGEKVVVRV | L220T |
| | | | TDERLVRLRDGTRSYFGAFMV | |
| LIGHT- | 63 | mLTβR+, | DGPAGSWEQLIQERRSHEVNPAAHLTGANSSLTGSGGPLLWETQLGLAF | T161G |
| 63 | | hLTβR+, | LRGLSYHDGALVVTKAGYYYIYSKVQLGGVGCPLGLASGITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 64 | mLTβR+, | DGPAGSWEQLIQERRSHEVNPAAHLTGANSSLTGSGGPLLWETQLGLAF | T161P |
| 64 | | hLTβR+, | LRGLSYHDGALVVTKAGYYYIYSKVQLGGVGCPLGLASPITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 65 | mLTβR+, | DGPAGSWEQLIQERRSHEVNPAAHLTGANSSLTGSGGPLLWETQLGLAF | S160G |
| 65 | | hLTβR+, | LRGLSYHDGALVVTKAGYYYIYSKVQLGGVGCPLGLAGTITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 66 | mLTβR+, | DGPAGSWEQLIQERRSHEVNPAAHLTGANSSLTGSGGPLLWETQLGLAF | S160G |
| 66 | | hLTβR+, | LRGLSYHDGALVVTKAGYYYIYSKVQLGGVGCPLGLAGTITHGLYKRTP | L220T |
| | | hHVEM+ | RYPEELELLVSQQSPCGRATSSSRVWWDSSFLGGVVHLEAGEKVVVRVT | L227T |
| | | | DERLVRTRDGTRSYFGAFMV | |
| LIGHT- | 67 | mLTβR+, | DGPAGSWEQLIQERRSHEVNPAAHLTGANSSLTGSGGPLLWETQLGLAF | T161S |
| 67 | | hLTβR+, | LRGLSYHDGALVVTKAGYYYIYSKVQLGGVGCPLGLASSITHGLYKRTP | L220S |
| | | hHVEM+ | RYPEELELLVSQQSPCGRATSSSRVWWDSSFLGGVVHLEAGEKVVVRVS | L227T |
| | | | DERLVRTRDGTRSYFGAFMV | |
| LIGHT- | 68 | mLTβR+, | DGPAGSWEQLIQERRSHEVNPAAHLTGANSSLTGSGGPLLWETQLGLAF | S160G |
| 68 | | hLTβR+, | LRGLSYHDGALVVTKAGYYYIYSKVQLGGVGCPLGLAGGITHGLYKRT | T161G |
| | | hHVEM+ | PRYPEELELLVSQQSPCGRATSSSRVWWDSSFLGGVVHLEAGEKVVVRV | L220S |
| | | | SDERLVRTRDGTRSYFGAFMV | L227T |
| LIGHT- | 69 | mLTβR+, | DGPAGSWEQLIQERRSHEVNPAAHLTGANSSLTGSGGPLLWETQLGLAF | S160G |
| 69 | | hLTβR+, | LRGLSYHDGALVVTKAGYYYIYSKVQLGGVGCPLGLAGPITHGLYKRTP | T161P |
| | | hHVEM+ | RYPEELELLVSQQSPCGRATSSSRVWWDSSFLGGVVHLEAGEKVVVRVS | L220S |
| | | | DERLVRTRDGTRSYFGAFMV | L227T |
| LIGHT- | 70 | mLTBR+, | DGPAGSWEQLIQERRSHEVNPAAHLTGANSSLTGSGGPLLWETNLGLAF | Q117N |
| 70 | | hLTβR+, | LRGLSYHDGALVVTKAGYYYIYSKVQLGGVGCPLGLASGITHGLYKRTP | T161G |
| | | hHVEM+ | RYPEELELLVSQQSPCGRATSSSRVWWDSSFLGGVVHLEAGEKVVVRVS | L220S |
| | | | DERLVRTRDGTRSYFGAFMV | L227T |
| LIGHT- | 71 | mLTβR+, | DGPAGSWEQLIQERRSHEVNPAAHLTGANSSLTGSGGPLLWETNLGLAF | Q117N |
| 71 | | hLTβR+, | LRGLSYHDGALVVTKAGYYYIYSKVQLGGVGCPLGLASPITHGLYKRTP | T161P |
| | | hHVEM+ | RYPEELELLVSQQSPCGRATSSSRVWWDSSFLGGVVHLEAGEKVVVRVS | L220S |
| | | | DERLVRTRDGTRSYFGAFMV | L227T |
| LIGHT- | 72 | mLTβR+, | DGPAGSWEQLIQERRSHEVNPAAHLTGANSSLTGSGGPLLWETNLGLAF | Q117N |
| 72 | | hLTβR+, | LRGLSYHDGALVVTKAGYYYIYSKVQLGGVGCPLGLAGTITHGLYKRTP | S160G |
| | | hHVEM+ | RYPEELELLVSQQSPCGRATSSSRVWWDSSFLGGVVHLEAGEKVVVRVS | L220S |
| | | | DERLVRTRDGTRSYFGAFMV | L227T |
| LIGHT- | 73 | mLTβR+, | DGPAGSWEQLIQERRSHEVNPAAHLTGANSSLTGSGGPLLWETELGLAF | Q117E |
| 73 | | hLTβR+, | LRGLSYHDGALVVTKAGYYYIYSKVQLGGVGCPLGLASGITHGLYKRTP | T161G |
| | | hHVEM+ | RYPEELELLVSQQSPCGRATSSSRVWWDSSFLGGVVHLEAGEKVVVRVS | L220S |
| | | | DERLVRTRDGTRSYFGAFMV | L227T |
| LIGHT- | 74 | mLTβR+, | DGPAGSWEQLIQERRSHEVNPAAHLTGANSSLTGSGGPLLWETNLGLAF | Q117N |
| 74 | | hLTβR+, | LRGLSYHDGALVVTKAGYYYIYSKVQLGGVGCPLGLAGGITHGLYKRT | S160G |
| | | hHVEM+ | PRYPEELELLVSQQSPCGRATSSSRVWWDSSFLGGVVHLEAGEKVVVRV | T161G |
| | | | SDERLVRTRDGTRSYFGAFMV | L220S |
| | | | | L227T |
| LIGHT- | 75 | mLTβR+ | DGPAGSWEQLIQERRSHEVNPAAHLTGANSSLTGSGGPLLWETNLGLAF | Q117N |
| 75 | | hLTβR+, | LRGLSYHDGALVVTKAGYYYIYSKVQLGGVGCPLGLAGPITHGLYKRTP | S160G |
| | | hHVEM+ | RYPEELELLVSQQSPCGRATSSSRVWWDSSFLGGVVHLEAGEKVVVRVS | T161P |
| | | | DERLVRTRDGTRSYFGAFMV | L220S |
| | | | | L227T |
| LIGHT- | 77 | mLTβR+, | DGPAGSWEQLIQERRSHEVNPAAHLTGANSSLTGSGGPLLWETELGLAF | Q117E |
| 77 | | hLTβR+, | LRGLSYHDGALVVTKAGYYYIYSKVQLGGVGCPLGLAGPITHGLYKRTP | S160G |
| | | hHVEM+ | RYPEELELLVSQQSPCGRATSSSRVWWDSSFLGGVVHLEAGEKVVVRVS | T161P |
| | | | DERLVRTRDGTRSYFGAFMV | L220S |
| | | | | L227T |
| LIGHT- | 78 | mLTβR+, | DGPAGSWEQLIQERRSHEVNPAAHLTGANSSLTGSGGPLLWETNLGLAF | Q117N |
| 78 | | hLTβR+, | LRGLSYHDGALVVTKAGYYYIYSKVQLGGVGCPLGLAGSITHGLYKRTP | S160G |
| | | hHVEM+ | RYPEELELLVSQQSPCGRATSSSRVWWDSSFLGGVVHLEAGEKVVVRVS | T161S |
| | | | DERLVRTRDGTRSYFGAFMV | L220S |
| | | | | L227T |
| LIGHT- | 79 | mLTβR+, | DGPAGSWEQLIQERRSHEVNPAAHLTGANSSLTGSGGPLLWETELGLAF | Q117E |
| 79 | | hLTβR+, | LRGLSYHDGALVVTKAGYYYIYSKVQLGGVGCPLGLAGSITHGLYKRTP | S160G |
| | | hHVEM+ | RYPEELELLVSQQSPCGRATSSSRVWWDSSFLGGVVHLEAGEKVVVRVS | T161S |
| | | | DERLVRTRDGTRSYFGAFMV | L220S |
| | | | | L227T |
| LIGHT- | 80 | mLTβR+, | DGPAGSWEQLIQERRSHEVNPAAHLTGANSSLTGSGGPLLWETNLGLAF | Q117N |
| 80 | | hLTβR+, | LRGLSYHDGALVVTKAGYYYIYSKVQLGGVGCPLGLAGGITHGLYKRT | S160G |
| | | hHVEM+ | PRYPEELELLVSQQSPCGRATSSSRVWWDSSFLGGVVHLEAGEKVVVRV | T161G |
| | | | SDSRLVRTRDGTRSYFGAFMV | L220S |
| | | | | E222S |
| | | | | L227T |
| LIGHT- | 81 | mLTβR+, | DGPAGSWEQLIQERRSHEVNPAAHLTGANSSLTGSGGPLLWETNLGLAF | Q117N |
| 81 | | hLTβR+, | LRGLSYHDGALVVTKAGYYYIYSKVQLGGVGCPLGLAGPITHGLYKRTP | S160G |
| | | hHVEM+ | RYPEELELLVSQQSPCGRATSSSRVWWDSSFLGGVVHLEAGEKVVVRVS | T161P |
| | | | DSRLVRTRDGTRSYFGAFMV | L220S |
| | | | | E222S |
| | | | | L227T |
| LIGHT- | 82 | mLTβR+, | DGPAGSWEQLIQERRSHEVNPAAHLTGANSSLTGSGGPLLWETELGLAF | Q117E |
| 82 | | hLTβR+, | LRGLSYHDGALVVTKAGYYYIYSKVQLGGVGCPLGLAGGITHGLYKRT | S160G |
| | | hHVEM+ | PRYPEELELLVSQQSPCGRATSSSRVWWDSSFLGGVVHLEAGEKVVVRV | T161G |
| | | | SDSRLVRTRDGTRSYFGAFMV | L220S |
| | | | | E222S |
| | | | | L227T |
| LIGHT- | 83 | mLTβR+, | DGPAGSWEQLIQERRSHEVNPAAHLTGANSSLTGSGGPLLWETELGLAF | Q117E |
| 83 | | hLTβR+, | LRGLSYHDGALVVTKAGYYYIYSKVQLGGVGCPLGLAGPITHGLYKRTP | S160G |
| | | hHVEM+ | RYPEELELLVSQQSPCGRATSSSRVWWDSSFLGGVVHLEAGEKVVVRVS | T161P |
| | | | DSRLVRTRDGTRSYFGAFMV | L220S |
| | | | | E222S |
| | | | | L227T |
| LIGHT- | 84 | mLTβR+, | DGPAGSWEQLIQERRSHEVNPAAHLTGANSSLTGSGGPLLWETNLGLAF | S160G |
| 84 | | hLTβR+, | LRGLSYHDGALVVTKAGYYYIYSKVQLGGVGCPLGLAGSITHGLYKRTP | T161S |
| | | hHVEM+ | RYPEELELLVSQQSPCGRATSSSRVWWDSSFLGGVVHLEAGEKVVVRVS | L220S |
| | | | DSRLVRTRDGTRSYFGAFMV | E222S |
| | | | | L227T |
| LIGHT- | 85 | mLTβR+, | DGPAGSWEQLIQERRSHEVNPAAHLTGANSSLTGSGGPLLWETELGLAF | Q117E |
| 85 | | hLTβR+, | LRGLSYHDGALVVTKAGYYYIYSKVQLGGVGCPLGLAGSITHGLYKRTP | S160G |
| | | hHVEM+ | RYPEELELLVSQQSPCGRATSSSRVWWDSSFLGGVVHLEAGEKVVVRVS | T161S |
| | | | DSRLVRTRDGTRSYFGAFMV | L220S |
| | | | | E222S |
| | | | | L227T |
| LIGHT- | 89 | mLTβR+, | DGPAGSWEQLIQERRSHEVNPAAHLTGANSSLTGSGGPLLWETQLGLAFL | G150R |
| 86 | | hLTβR+, | RGLSYHDGALVVTKAGYYYIYSKVQLRGVGCPLGLARTITHGLYKRTPR | S160R |
| | | hHVEM+ | YPEELELLVSQQSPCGRATSSSRVWWDSSFLGGVVHLEAGEKVVVRVSD | L220S |
| | | | ERLVRLRDGTRSYFGAFMV | |
| LIGHT- | 90 | mLTβR+, | DGPAGSWEQLIQERRSHEVNPAAHLTGANSSLTGSGGPLLWETQLGLAFL | G150R |
| 87 | | hLTβR+, | RGLSYHDGALVVTKAGYYYIYSKVQLRGVGCPLGLARTITHGLYKRTPR | S160R |
| | | hHVEM+ | YPEELELLVSQQSPCGRATSSSRVWQDSSFLGGVVHLEAGEKVVVRVSDE | W198Q |
| | | | RLVRLLDGTRSYFGAFMV | L220S |
| | | | | R228L |
| LIGHT- | 91 | mLTβR+, | DGPAGSWEQLIQERRSHEVNPTAHLTGANSSLTGSGGPLLWETQLGLAFL | A95T |
| 88 | | hLTβR+, | RGLSYHDGALVVTKAGYYYIYSKVQLRGVGCPLGLARTITHGLYKRTPR | G150R |
| | | hHVEM+ | YLEELELLVSQQSPCGRATSSSRVWQDSSFLGGVVHLEAGEKVVVRVSDE | S160R |
| | | | RLVRLLDGTRSYFGAFMV | P174L |
| | | | | W198Q |
| | | | | L220S |
| | | | | R228L |
| LIGHT- | 92 | mLTβR+, | DGPAGSWEQLIQERRSHEVNPTAHLTGANSSLTGSGGPLLWETQLGLAFL | G150R |
| 89 | | hLTβR+, | RGLSYHDGALVVTKAGYYYIYSKVQLRGVGCPLGLARTITHGLYKRTPR | S160R |
| | | hHVEM+ | YLEELELLVSQQSPCGRATSSSRVWQDSSFLGGVVHLEAGEKVVVRVLD | P174L |
| | | | ERLVRLLDGTRSYFGAFMV | W198Q |
| | | | | R228L |
| LIGHT- | 93 | mLTβR+, | DGPAGSWEQLIQERRSHEVNPAAHLTGANSSLTGSGGPLLWETQLGLAFL | S160G |
| 90 | | hLTβR+, | RGLSYHDGALVVTKAGYYYIYSKVQLGGVGCPLGLAGGITHGLYKRTPR | T161G |
| | | hHVEM+ | YPEELELLVSQQSPCGRATSSSRVWWDSSFLGGVVHLEAGEKVVVRVTD | L220T |
| | | | SRLVRLRDGTRSYFGAFMV | E222S |

+: strong binding; −: no binding; +/−: weak binding; N.A: not available/no data

TABLE 4
Amino acid mutations and the positions be mutated

| POSI- | | | POSI- | | |
|---|---|---|---|---|---|
| TION | WT | MUTANT | TION | WT | MUTANT |
| 101 | A | D | 166 | L | M |
| 102 | N | R | 175 | E | K |
| 103 | S | N | 184 | Q | R |
| 104 | S | P | 189 | R | S |
| 105 | L | P | 190 | A | T, V |
| 116 | T | S | 198 | W | Q |
| 117 | Q | E, N, H, R | 202 | F | Y |
| 120 | L | P, Q | 208 | H | Y, R |
| 126 | L | M | 213 | E | D |
| 135 | V | I | 214 | K | E |
| 136 | T | S | 220 | L | S, T, M, R, Q, N |
| 150 | G | A, S, R, | 221 | D | G |
| 152 | V | M | 222 | E | S, K, D, Q, N |
| 156 | L | P | 223 | R | H |
| 158 | L | M, Q, P, | 227 | L | T, M |
| 160 | S | T, N, G, H, R, A | 228 | R | L |
| 161 | T | S, N, G, P | 232 | R | H |
| 95 | A | T | 174 | P | L |
| 155 | P | R | 157 | G | S |

### Example 5: Expression and Purification of LIGHT Muteins

DNA fragments of human LIGHT mutants were synthesized by Genewiz and cloned into the pCI vector (E1731, Promega) with an N-terminal His tag. The constructs were transfected into Expi293F cells (A14527, ThermoFisher) and cultured in suspension culture for about five days. The supernatant was harvested by centrifugation at 7,000 RPM for 20 minutes at 4° C. and filtered through a 0.22 μm filter. Subsequently, the filtered supernatant was incubated with magnetic nickel-NTA beads (Ni Smart Beads 6FF, Smart-Lifesciences, China) for 1 hour. After washing with 10 CV (column volume) of PBS, the LIGHT muteins were eluted using PBS containing 300 mM imidazole and 0.3 M NaCl. Finally, the eluted protein was dialyzed with PBS (pH 6.5) and 5% glycerol.

### Example 6: Affinities of LIGHT Muteins with HVEM and LTβR

Nunc MaxiSorp 96-well plates (Thermofisher) were coated with target proteins (1 μg/ml), and then blocked with 2% BSA-PBS buffer for one hour. Serial dilutions of the LIGHT muteins were added with a maximum concentration of 200 ng/ml. The plates were incubated for one hour, washed four times with PBST, and then incubated with mouse anti-His tag antibody (#105327, Sino Biological) for 1 hour. The plates were further incubated with HRP-conjugated goat anti-mouse secondary antibody (#SSA006, Sino Biological) before being washed three times with PBST and treated with TMB substrate (#34029, ThermoFisher). The plates were read at 450 nM on a SpectraMax M5 microplate reader (Molecule Devices). The ELISA results were analyzed using GraphPad Prism 9.0 software and the EC50s (half maximal effective concentration) were summarized in Table 5-1 and Table 5-2.

TABLE 5-1
Calculated EC50 results of LIGHT muteins.

| | EC50 (ng/ml) | | |
|---|---|---|---|
| Name | human LTßR | mouse LTßR | human HVEM |
| LIGHT (74-240) | 220.3 | weak (the EC50 is too | weak (the EC50 is too |
| (wt) | | low to fit accurately) | low to fit accurately) |
| LIGHT-1 | 49.31 | 60.5 | — |
| LIGHT-2 | 76.2 | weak (similar to wt) | — |
| LIGHT-3 | 43.64 | 40.06 | — |
| LIGHT-5 | 53.81 | weak (similar to wt) | — |
| LIGHT-9 | 28.21 | 22.37 | — |
| LIGHT-11 | 39.49 | 23.33 | — |
| LIGHT-15 | 74.83 | 139.4 (similar to wt) | 59.73 |
| LIGHT-17 | 80.48 | 44.16 | 51.09 |
| LIGHT-21 | 71.46 | 134.1 | 42.19 |
| LIGHT-22 | 68.59 | 62.45 | 47.07 |
| LIGHT-29 | 45.21 | 20.94 | 42.77 |
| LIGHT-37 | 43.38 | 67.83 | 37.02 |
| LIGHT-46 | 67.47 | Weak (similar to wt) | 97.82 |
| LIGHT-52 | 57.73 | 27.87 | 19.01 |
| LIGHT-7 | 109.4 | 156.3 | — |
| LIGHT-10 | 44.07 | 80.35 | 46.7 |
| LIGHT-23 | 89.16 | 91.6 | 58.8 |
| LIGHT-26 | 72.67 | 48.3 | 48.4 |
| LIGHT-28 | 81.1 | 256.2 (similar to wt) | 87.3 |
| LIGHT-31 | 86.65 | 74.71 | 51 |
| LIGHT-32 | 247.4 (similar to wt) | 63.18 | Weak (similar to wt) |
| LIGHT-39 | 54.18 | 23.94 | 49 |
| LIGHT-47 | 228.3 (similar to wt) | 70.58 | Weak (similar to wt) |
| LIGHT-48 | 755.8 (similar to wt) | 419.9 weak (similar to wt) | Weak (similar to wt) |
| LIGHT-14 | weak (similar to wt) | 24.92 | 30.0 |
| LIGHT-16 | 160 (similar to wt) | weak (similar to wt) | 73.8 |
| LIGHT-18 | 89.41 | 41.28 | 44.7 |
| LIGHT-19 | weak (similar to wt) | 468.2 (similar to wt) | 68.1 |
| LIGHT-20 | 52.14 | weak (similar to wt) | 49.6 |
| LIGHT-24 | weak (similar to wt) | weak (similar to wt) | 63.2 |
| LIGHT-25 | weak (similar to wt) | 32.27 | 54.2 |
| LIGHT-27 | 82.71 | 64.19 | 50.4 |
| LIGHT-30 | — | — | 156.7 |
| LIGHT-33 | 61.15 | weak (similar to wt) | 25.6 |
| LIGHT-34 | 58.99 | 44.64 | 20.2 |
| LIGHT-35 | 70.32 | 38.3 | 41.6 |
| LIGHT-36 | 190.1 (similar to wt) | 270.3 weak (similar to wt) | 136.7 |
| LIGHT-40 | 95.9 | weak (similar to wt) | 50.3 |
| LIGHT-41 | 108.5 | 31.05 | — |
| LIGHT-42 | 63.86 | 24.99 | 81.4 |
| LIGHT-45 | 36.19 | 26.44 | — |

wt means wild type LIGHT (74-240) shown in SEQ ID NO: 87
“—” means no binding was detected by phage ELISA

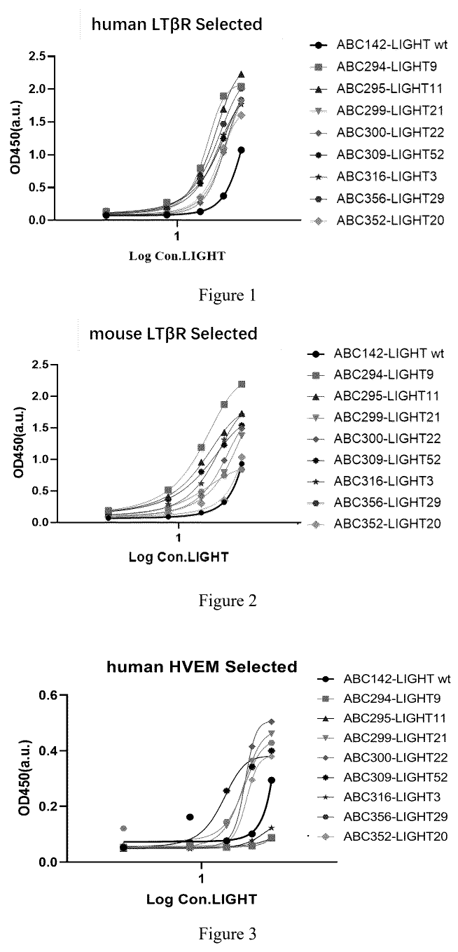

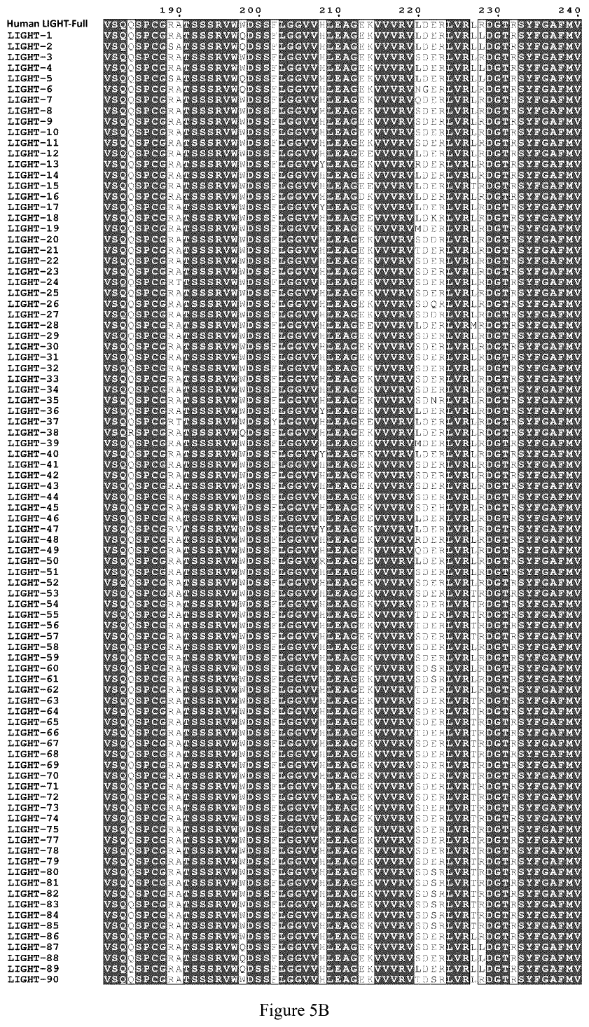

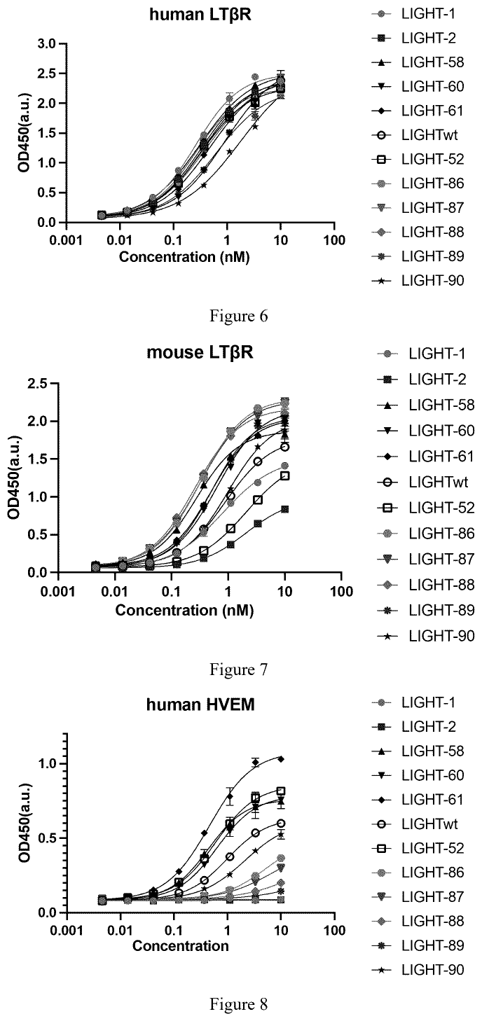

Most LIGHT muteins showed higher affinity to LTβRs (both human and mouse) than wild-type human LIGHT (74-240). Some LIGHT muteins, such as LIGHT-1, LIGHT-3, LIGHT-9 and LIGHT-11, had reduced affinity to human HVEM compared to wild type (FIGS. 1-3, FIGS. 6-8 and FIG. 10).

In addition to HVEM and LTβR, LIGHT also interacts with a decoy receptor, DcR3, which lacks the transmembrane and cytoplasmic segments. This interaction has the potential to disrupt the signaling pathways by sequestering LIGHT away from HVEM and LTβR. While DcR3 expression is typically low in healthy human tissues, it is often significantly upregulated in cancer patients (Wu et al., 2003; Yoo et al., 2022).

To test whether the muteins can bind to DcR3, the recombinant DcR3 protein was generated by linking human DcR3 residues 33-300 to the N-terminal of rabbit Fc. The recombinant proteins were expressed in Expi293F and affinity-purified using protein A beads, as previously described. Human DcR3 proteins were immobilized onto maxiSorp 96-well ELISA plates (Thermo Fisher) at a concentration of 0.5 μg/mL and then blocked with 2% BSA-PBS buffer for one hour. The LIGHT variants were added to the plates at various dilutions, with a maximum concentration of 10 nM. The plates were incubated for one hour, washed four times with PBST, and then incubated with mouse anti-His tag antibody (#105327, Sino Biological) for 1 hour. The plates were further incubated with HRP-conjugated goat anti-mouse secondary antibody (#SSA006, Sino Biological) before being washed three times with PBST and treated with TMB substrate (#34029, ThermoFisher). The plates were read at 450 nM on a SpectraMax M5 microplate reader (Molecule Devices). The ELISA results were analyzed using GraphPad Prism 9.0 software and the EC50s (half maximal effective concentration) were summarized in Table 5-2.

TABLE 5-2
EC50s (nM) for LIGHT binding
to LTßR, HVEM and human DcR3.

| | human | mouse | human | Human |
|---|---|---|---|---|
| Name | LTβR | LTβR | HVEM | DcR3 |
| LIGHT-1 | 0.2616 | 0.8003 | N.D. | N.D. |
| LIGHT-2 | 0.3282 | 2.055 | N.D. | N.D. |
| LIGHT-58 | 0.3094 | 0.2727 | 0.4316 | 0.1839 |
| LIGHT-60 | 0.9191 | 0.6303 | 0.639 | 0.3107 |
| LIGHT-61 | 0.4817 | 0.4981 | 0.4598 | 0.2135 |
| LIGHT (74-240) (wt) | 0.3646 | 0.8854 | 0.992 | 0.2469 |
| LIGHT-52 | 0.3304 | 2.071 | 0.5991 | 0.1904 |
| LIGHT-86 | 0.3355 | 0.3266 | 5.273 | 25.87 |
| LIGHT-87 | 0.4132 | 0.3148 | 6.683 | N.D. |
| LIGHT-88 | 0.2641 | 0.2624 | N.D. | N.D. |
| LIGHT-89 | 0.625 | 0.503 | N.D. | N.D. |
| LIGHT-90 | 1.776 | 0.978 | 2.119 | 0.6924 |

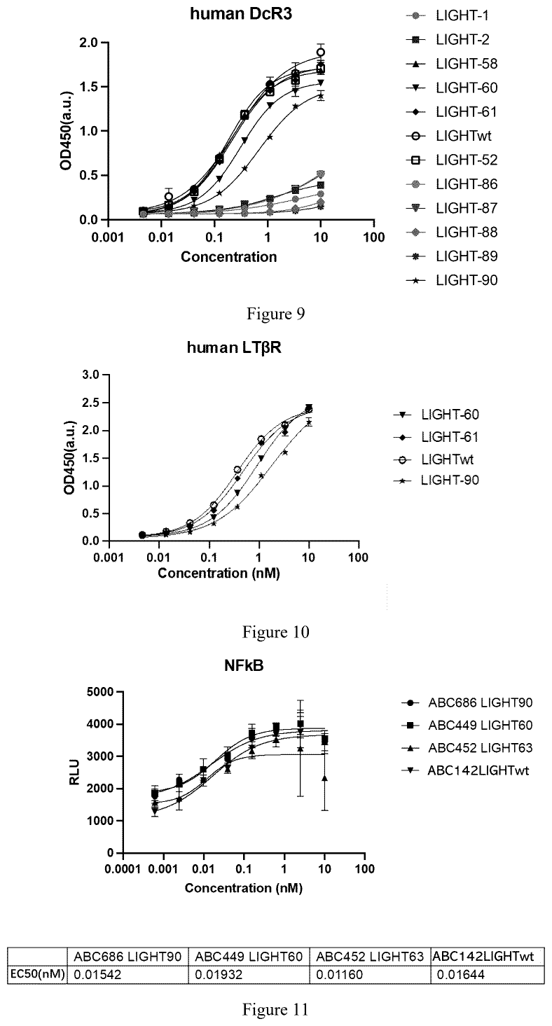

As shown in Table 5-2 and FIG. 9, the affinity of LIGHT-1, LIGHT-2, LIGHT-86, LIGHT-87, LIGHT-88, LIGHT-89 towards DcR3 had been observed to be reduced.

### Example 7: Functional Assay

HeLa—NF-κB-reporter cell line was generated by transfecting the cells with the pNL3.2.NF—κB-RE [NlucP/NF—κB-RE/Hygro] vector (Promega #N1111) using lipofectamine 3000. After three days, the cells were treated with hygromycin B (Sigma) and cultured for 14 days in 37° C., 5% CO2. The resulting HeLa—NF-κB reporter cells were used to evaluate the downstream signaling of LTβR activation by the treatment of LIGHT muteins. HeLa—NF-κB reporter cells were exposed to serial dilutions of LIGHT muteins. After 24 hours, the cells were lysed (Promega #E397A) and the luciferase activity was measured with a SpectraMax M5 microplate reader (Molecule Devices) using Promega #E4500.

As shown in FIG. 11, the activity of stimulating hLTβR of LIGHT90, LIGHT60 and LIGHT63 were consistent with binding affinity.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

What is claimed is:

1. A LIGHT mutein, which is selected from the group consisting a protein having more than 80%, 85%, 90%, 95%, 96%, 97%, 98%, 99% homology to the sequence set forth in SEQ ID NO: 86, 87 or 88, and with at least one amino acid difference compared with the amino acid sequence set forth in SEQ ID NO: 86, 87 or 88.

2. The LIGHT mutein according to claim 1, which is selected from the group consisting a protein having more than 80%, 85%, 90%, 95%, 96%, 97%, 98%, 99% homology to the sequence from position of SEQ ID NO: 87, and with at least one amino acid difference compared with the amino acid sequence set forth in SEQ ID NO: 87.

3. The LIGHT mutein according to claim 1 or 2, comprising one or more amino acid mutations as compared with the amino acid sequence set forth in SEQ ID NO: 87,
wherein the amino acid mutations selected from one or more positions selected from the group consisting of 95, 101, 102, 103, 104, 116, 117, 120, 126, 135, 136, 150, 152, 155, 156, 157, 158, 160, 161, 166, 174, 175, 184, 189, 190, 198, 202, 208, 213, 214, 220, 221, 222, 223, 227, 228, 232, and the combination thereof,
wherein the positions are defined with reference to SEQ ID NO: 87, and the position of the first amino acid of SEQ ID NO: 87 is defined as position 74.

4. The LIGHT mutein according to any one of claims 1-3, wherein the one or more amino acid mutations are selected from the group consisting of Q117E/Q117N/Q117H, G150A/G150S, S160G/S160N/S160T/S160H/S160A, T161G/T161P/T161S/T161N, W198Q, K214E, L220S/L220N/L220T/L220M/L220R/L220Q, E222K/E222S/E222Q/E222D/E222N, R228L, R232H, and the combination thereof,
wherein the positions are defined with reference to SEQ ID NO: 87, and the position of the first amino acid of SEQ ID NO: 87 is defined as position 74.

5. The LIGHT mutein according to any one of claims 1-4, wherein the one or more amino acid mutations are selected from the group consisting of A95T, A101D, N102R, S103N, S104P, L105P, T116S, Q117E/Q117N/Q117H/Q117R, L126M, V135I, T136S, G150A/G150S/G150R, V152M, P155R, L156P, G157S, L158Q/L158P/L158M, S160G/S160N/S160T/S160A, T161G/T161P/T161S/T161N, L166M, P174L, E175K, Q184R, R189S, A190T/A190V, W198Q, F202Y, H208Y/H208R, E213D, K214E, L220S/L220N/L220T/L220R/L220M, D221G, E222K/E222S/E222Q/E222D/E222N, L227T/L227M, R228L, R232H and the combination thereof,
wherein the positions are defined with reference to SEQ ID NO: 87, and the position of the first amino acid of SEQ ID NO: 87 is defined as position 74.

6. The LIGHT mutein according to any one of claims 1-5, wherein the LIGHT mutein comprises any one of mutation combinations: V152M, W198Q and R228L; R189S, W198Q and R228L; G150A and L220S; V152M, T161P and R228L; T161P, R189S, W198Q and R228L; W198Q, L220N and D221G; P155R, L220Q and R232H; G150S, S160G and L220S; G150S, T161G and L220S; G150S, T161P and L220S; G150S and L220S; L158Q and K214E; S104P, G157S, H208Y and L220R; L158Q and L166M; Q117E, E175K, K214E and L227T; Q117H, L158M and E213D; H208Y and Q117N; L158Q, K214E and E222K; L156P, S160G and L220M; S160A, L220S and E222D; S160G, T161P and L220T; S160G, T161S and L220S; S160G and L220S; S160G, A190T and L220S; S160H and L220S; S160H, L220S and E222Q; S160N, T161S, L220S and E222D; S160N, K214E and L227M; S160N and L220S; S160R, T161N and L220S; S160R, T161S and L220S; S160R and L220S; T161P and L220S; T161S and L220S; T161S, L220S and E222N; H208Y; A190T, F202Y and K214E; Q184R and W198Q; L158P, L166M and L220M; L156P and H208Y; G150R, S160G and L220S; G150R and L220S; G150R, S160T and L220S; G150R, L158P and L220S; G150S, S160P, T161S, L220S and R223H; L126M and K214E; L126M, A190V and H208Y; L120P and L220R; L105P, Q117R and L220Q; N102R, L120Q, H208R and K214E; A101D, S160N and L220S; L220S; Q117N, L220S and L227T; Q117E, L220S and L227T; Q117N, L220T and L227T; Q117E, L220T and L227T; L220S and L227T; S160G, T161G and L220S; S160G, T161P and L220S; S160G, T161G, L220S and E222S; S160G, T161P, L220S and E222S; S160G, T161G and L220T; T161G, L220S and L227T; T161P, L220S and L227T; S160G, L220S and L227T; S160G, L220T and L227T; T161S, L220S and L227T; S160G, T161G, L220S and L227T; S160G, T161P, L220S and L227T; Q117N, T161G, L220S and L227T; Q117N, T161P, L220S and L227T; Q117N, S160G, L220S and L227T; Q117E, T161G, L220S and L227T; Q117N, S160G, T161G, L220S and L227T; Q117N, S160G, T161P, L220S and L227T; Q117E, S160G, T161P, L220S and L227T; Q117N, S160G, T161S, L220S and L227T; Q117E, S160G, T161S, L220S and L227T; Q117N, S160G, T161G, L220S, E222S and L227T; Q117N, S160G, T161P, L220S, E222S and L227T; Q117E, S160G, T161G, L220S, E222S and L227T; Q117E, S160G, T161P, L220S, E222S and L227T; S160G, T161S, L220S, E222S and L227T; Q117E, S160G, T161S, L220S, E222S and L227T; G150R, S160R and L220S; G150R, S160R, W198Q, L220S and R228L; A95T, G150R, S160R, P174L, W198Q, L220S and R228L; G150R, S160R, P174L, W198Q and R228L; or S160G, T161G, L220T and E222S.

7. The LIGHT mutein according to any one of claims 1-6, comprising the amino acid sequence with at least 95%, 96%, 97%, 98% or 99% homology to amino acid sequence set forth in any one of SEQ ID NOs: 1-75, 77-85 and 89-93.

8. A LTβR binding LIGHT mutein, comprising an amino acid sequence set forth in any one of SEQ ID NOs: 1-75, 77-85 and 89-93.

9. The LTβR binding LIGHT mutein according to claim 8, wherein the LIGHT mutein is HVEM non-binding, the LIGHT mutein comprises the amino acid sequence set forth in any one of SEQ ID NOs: 1-11.

10. The LTβR binding LIGHT mutein according to claim 8 or 9, the LIGHT mutein is HVEM binding, the LIGHT mutein comprises an amino acid sequence set forth in any one of SEQ ID NOs: 12-75, SEQ ID NOs: 77-85 and SEQ ID NOs: 89-93.

11. The LTβR binding LIGHT mutein according to any one of claims 8-10, wherein the LIGHT mutein binds to DcR3 with reduced affinity, compared to wildtype LIGHT, the LIGHT mutein comprises an amino acid sequence set forth in any one of SEQ ID NOs: 1, 2, 52, 58, 61 and 89-93.

12. An isolated polynucleotide encoding the LIGHT mutein according to any one of claims 1-11.

13. An isolated vector comprising the polynucleotide according to claim 12.

14. A host cell comprising the isolated polynucleotide according to claim 12 or the isolated vector according to claim 13.

15. A pharmaceutical composition comprising the LIGHT mutein according to any one of claims 1-11 or the isolated polynucleotide according to claim 12 or the isolated vector according to claim 13 or the host cell according to claim 14, and a pharmaceutically acceptable carrier.

16. Use of the LIGHT mutein according to any one of claims 1-11 or the isolated polynucleotide according to claim 12 or the isolated vector according to claim 13 or the host cell according to claim 14, or the pharmaceutical composition according to claim 15 in the manufacture of a therapeutic agent for diagnosing, preventing, or treating a disease, disorder, or condition.

17. A method of diagnosing, preventing or treating a disease, disorder, or condition in a subject in need thereof, comprising administrating to the subject a therapeutically effective amount of the LIGHT mutein according to any one of claims 1-11 or the isolated polynucleotide according to claim 12 or the isolated vector according to claim 13 or the host cell according to claim 14 or the pharmaceutical composition according to claim 15.


SEQUENCE LISTING (93 sequences)

SEQ ID NO: 1 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGMGCPLGLASTITHGLYKRTPRYPEELELLVSQQSPCGRATSSSRVWQDSSFLGGVVHLEAGEKVVVRVLDERLVRLLDGTRSYFGAFMV

SEQ ID NO: 2 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGSATSSSRVWQDSSFLGGVVHLEAGEKVVVRVLDERLVRLLDGTRSYFGAFMV

SEQ ID NO: 3 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLAGVGCPLGLASTITHGLYKRTPRYPEELELLVSQQSPCGRATSSSRVWWDSSFLGGVVHLEAGEKVVVRVSDERLVRLRDGTRSYFGAFMV

SEQ ID NO: 4 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGMGCPLGLASPITHGLYKRTPRYPEELELLVSQQSPCGRATSSSRVWWDSSFLGGVVHLEAGEKVVVRVLDERLVRLLDGTRSYFGAFMV

SEQ ID NO: 5 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASPITHGLYKRTPRYPEELELLVSQQSPCGSATSSSRVWQDSSFLGGVVHLEAGEKVVVRVLDERLVRLLDGTRSYFGAFMV

SEQ ID NO: 6 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWQDSSFLGGVVHLEAGEKVVVRVNGERLVRLRDGTRSYFGAFMV

SEQ ID NO: 7 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCRLGLASTITHGLYKRTPRYPEELELLVSQQSPCGRATSSSRVWWDSSFLGGVVHLEAGEKVVVRVQDERLVRLRDGTHSYFGAFMV

SEQ ID NO: 8 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLSGVGCPLGLAGTITHGLYKRTPRYPEELELLVSQQSPCGRATSSSRVWWDSSFLGGVVHLEAGEKVVVRVSDERLVRLRDGTRSYFGAFMV

SEQ ID NO: 9 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLSGVGCPLGLASGITHGLYKRTPRYPEELELLVSQQSPCGRATSSSRVWWDSSFLGGVVHLEAGEKVVVRVSDERLVRLRDGTRSYFGAFMV

SEQ ID NO: 10 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLSGVGCPLGLASPITHGLYKRTPRYPEELELLVSQQSPCGRATSSSRVWWDSSFLGGVVHLEAGEKVVVRVSDERLVRLRDGTRSYFGAFMV

SEQ ID NO: 11 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLSGVGCPLGLASTITHGLYKRTPRYPEELELLVSQQSPCGRATSSSRVWWDSSFLGGVVHLEAGEKVVVRVSDERLVRLRDGTRSYFGAFMV

SEQ ID NO: 12 - protein, 167 aa, synthetic construct
DGPAGSWEQLIQERRSHEVNPAAHLTGANNSLTGSGGPLLWETQLGLAFLRGLSYHDGALVVTKAGYYYIYSKVQLGGVGCPLGQASTITHGLYKRTPRYPEELELLVSQQSPCGRATSSSRVWWDSSFLGGVVHLEAGEEVVVRVLDERLVRLRDGTRSYFGAFMV

SEQ ID NO: 13 - protein, 167 aa, synthetic construct
DGPAGSWEQLIQERRSHEVNPAAHLTGANSPLTGSGGPLLWETQLGLAFLRGLSYHDGALVVTKAGYYYIYSKVQLGGVGCPLSLASTITHGLYKRTPRYPEELELLVSQQSPCGRATSSSRVWWDSSFLGGVVYLEAGEKVVVRVRDERLVRLRDGTRSYFGAFMV

SEQ ID NO: 14 - protein, 167 aa, synthetic construct
DGPAGSWEQLIQERRSHEVNPAAHLTGANSSLTGSGGPLLWESQLGLAFLRGLSYHDGALVVTKAGYYYIYSKVQLGGVGCPLGQASTITHGMYKRTPRYPEELELLVSQQSPCGRATSSSRVWWDSSFLGGVVHLEAGEKVVVRVLDERLVRLRDGTRSYFGAFMV

SEQ ID NO: 15 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLASTITHGLYKRTPRYPKELELLVSQQSPCGRATSSSRVWWDSSFLGGVVHLEAGEEVVVRVLDERLVRTRDGTRSYFGAFMV

SEQ ID NO: 16 - protein, 167 aa, synthetic construct
DGPAGSWEQLIQERRSHEVNPAAHLTGANSSLTGSGGPLLWETHLGLAFLRGLSYHDGALVVTKAGYYYIYSKVQLGGVGCPLGMASTITHGLYKRTPRYPEELELLVSQQSPCGRATSSSRVWWDSSFLGGVVHLEAGDKVVVRVLDERLVRLRDGTRSYFGAFMV

SEQ ID NO: 17 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLASTITHGLYKRTPRYPEELELLVSQQSPCGRATSSSRVWWDSSFLGGVVYLEAGEKVVVRVLDERLVRLRDGTRSYFGAFMV

SEQ ID NO: 18 - protein, 167 aa, synthetic construct
DGPAGSWEQLIQERRSHEVNPAAHLTGANSSLTGSGGPLLWETQLGLAFLRGLSYHDGALVITKAGYYYIYSKVQLGGVGCPLGQASTITHGLYKRTPRYPEELELLVSQQSPCGRATSSSRVWWDSSFLGGVVHLEAGEEVVVRVLDKRLVRLRDGTRSYFGAFMV

SEQ ID NO: 19 - protein, 167 aa, synthetic construct
DGPAGSWEQLIQERRSHEVNPAAHLTGANSSLTGSGGPLLWETQLGLAFLRGLSYHDGALVVSKAGYYYIYSKVQLGGVGCPPGLAGTITHGLYKRTPRYPEELELLVSQQSPCGRATSSSRVWWDSSFLGGVVHLEAGEKVVVRVMDERLVRLRDGTRSYFGAFMV

SEQ ID NO: 20 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAATITHGLYKRTPRYPEELELLVSQQSPCGRATSSSRVWWDSSFLGGVVHLEAGEKVVVRVSDDRLVRLRDGTRSYFGAFMV

SEQ ID NO: 21 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVTDERLVRLRDGTRSYFGAFMV

SEQ ID NO: 22 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGSITHGLYKRTPRYPEELELLVSQQSPCGRATSSSRVWWDSSFLGGVVHLEAGEKVVVRVSDERLVRLRDGTRSYFGAFMV

SEQ ID NO: 23 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGTITHGLYKRTPRYPEELELLVSQQSPCGRATSSSRVWWDSSFLGGVVHLEAGEKVVVRVSDERLVRLRDGTRSYFGAFMV

SEQ ID NO: 24 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGTITHGLYKRTPRYPEELELLVSQQSPCGRTTSSSRVWWDSSFLGGVVHLEAGEKVVVRVSDERLVRLRDGTRSYFGAFMV

SEQ ID NO: 25 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAHTITHGLYKRTPRYPEELELLVSQQSPCGRATSSSRVWWDSSFLGGVVHLEAGEKVVVRVSDERLVRLRDGTRSYFGAFMV

SEQ ID NO: 26 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAHTITHGLYKRTPRYPEELELLVSQQSPCGRATSSSRVWWDSSFLGGVVHLEAGEKVVVRVSDQRLVRLRDGTRSYFGAFMV

SEQ ID NO: 27 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLANSITHGLYKRTPRYPEELELLVSQQSPCGRATSSSRVWWDSSFLGGVVHLEAGEKVVVRVSDDRLVRLRDGTRSYFGAFMV

SEQ ID NO: 28 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLANTITHGLYKRTPRYPEELELLVSQQSPCGRATSSSRVWWDSSFLGGVVHLEAGEEVVVRVLDERLVRMRDGTRSYFGAFMV

SEQ ID NO: 29 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLANTITHGLYKRTPRYPEELELLVSQQSPCGRATSSSRVWWDSSFLGGVVHLEAGEKVVVRVSDERLVRLRDGTRSYFGAFMV

SEQ ID NO: 30 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLARNITHGLYKRTPRYPEELELLVSQQSPCGRATSSSRVWWDSSFLGGVVHLEAGEKVVVRVSDERLVRLRDGTRSYFGAFMV

SEQ ID NO: 31 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLARSITHGLYKRTPRYPEELELLVSQQSPCGRATSSSRVWWDSSFLGGVVHLEAGEKVVVRVSDERLVRLRDGTRSYFGAFMV

SEQ ID NO: 32 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLARTITHGLYKRTPRYPEELELLVSQQSPCGRATSSSRVWWDSSFLGGVVHLEAGEKVVVRVSDERLVRLRDGTRSYFGAFMV

SEQ ID NO: 33 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASPITHGLYKRTPRYPEELELLVSQQSPCGRATSSSRVWWDSSFLGGVVHLEAGEKVVVRVSDERLVRLRDGTRSYFGAFMV

SEQ ID NO: 34 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASSITHGLYKRTPRYPEELELLVSQQSPCGRATSSSRVWWDSSFLGGVVHLEAGEKVVVRVSDERLVRLRDGTRSYFGAFMV

SEQ ID NO: 35 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASSITHGLYKRTPRYPEELELLVSQQSPCGRATSSSRVWWDSSFLGGVVHLEAGEKVVVRVSDNRLVRLRDGTRSYFGAFMV

SEQ ID NO: 36 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVYLEAGEKVVVRVLDERLVRLRDGTRSYFGAFMV

SEQ ID NO: 37 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRTTSSSRVWWDSSYLGGVVHLEAGEEVVVRVLDERLVRLRDGTRSYFGAFMV

SEQ ID NO: 38 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQRSPCGRATSSSRVWQDSSFLGGVVHLEAGEKVVVRVLDERLVRLRDGTRSYFGAFMV

SEQ ID NO: 39 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGPASTITHGMYKRTPRYPEELELLVSQQSPCGRATSSSRVWWDSSFLGGVVHLEAGEKVVVRVMDERLVRLRDGTRSYFGAFMV

SEQ ID NO: 40 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPPGLASTITHGLYKRTPRYPEELELLVSQQSPCGRATSSSRVWWDSSFLGGVVYLEAGEKVVVRVLDERLVRLRDGTRSYFGAFMV

SEQ ID NO: 41 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGLAGTITHGLYKRTPRYPEELELLVSQQSPCGRATSSSRVWWDSSFLGGVVHLEAGEKVVVRVSDERLVRLRDGTRSYFGAFMV

SEQ ID NO: 42 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGLASTITHGLYKRTPRYPEELELLVSQQSPCGRATSSSRVWWDSSFLGGVVHLEAGEKVVVRVSDERLVRLRDGTRSYFGAFMV

SEQ ID NO: 43 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGLATTITHGLYKRTPRYPEELELLVSQQSPCGRATSSSRVWWDSSFLGGVVHLEAGEKVVVRVSDERLVRLRDGTRSYFGAFMV

SEQ ID NO: 44 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGPASTITHGLYKRTPRYPEELELLVSQQSPCGRATSSSRVWWDSSFLGGVVHLEAGEKVVVRVSDERLVRLRDGTRSYFGAFMV

SEQ ID NO: 45 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLSGVGCPLGLAPSITHGLYKRTPRYPEELELLVSQQSPCGRATSSSRVWWDSSFLGGVVHLEAGEKVVVRVSDEHLVRLRDGTRSYFGAFMV

SEQ ID NO: 46 - protein, 167 aa, synthetic construct
DGPAGSWEQLIQERRSHEVNPAAHLTGANSSLTGSGGPLLWETQLGLAFLRGMSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 47 - protein, 167 aa, synthetic construct
DGPAGSWEQLIQERRSHEVNPAAHLTGANSSLTGSGGPLLWETQLGLAFLRGMSYHDGALVVTKAGYYYIYSKVQLGGVGCPLGLASTITHGLYKRTPRYPEELELLVSQQSPCGRVTSSSRVWWDSSFLGGVVYLEAGEKVVVRVLDERLVRLRDGTRSYFGAFMV

SEQ ID NO: 48 - protein, 167 aa, synthetic construct
DGPAGSWEQLIQERRSHEVNPAAHLTGANSSLTGSGGPLLWETQLGPAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVRDERLVRLRDGTRSYFGAFMV

SEQ ID NO: 49 - protein, 167 aa, synthetic construct
DGPAGSWEQLIQERRSHEVNPAAHLTGANSSPTGSGGPLLWETRLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVQDERLVRLRDGTRSYFGAFMV

SEQ ID NO: 50 - protein, 167 aa, synthetic construct
DGPAGSWEQLIQERRSHEVNPAAHLTGARSSLTGSGGPLLWETQLGQAFLRGLSYHDGALVVTKAGYYYIYSKVQLGGVGCPLGLASTITHGLYKRTPRYPEELELLVSQQSPCGRATSSSRVWWDSSFLGGVVRLEAGEEVVVRVLDERLVRLRDGTRSYFGAFMV

SEQ ID NO: 51 - protein, 167 aa, synthetic construct
DGPAGSWEQLIQERRSHEVNPAAHLTGDNSSLTGSGGPLLWETQLGLAFLRGLSYHDGALVVTKAGYYYIYSKVQLGGVGCPLGLANTITHGLYKRTPRYPEELELLVSQQSPCGRATSSSRVWWDSSFLGGVVHLEAGEKVVVRVSDERLVRLRDGTRSYFGAFMV

SEQ ID NO: 52 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVSDERLVRLRDGTRSYFGAFMV

SEQ ID NO: 53 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVSDERLVRTRDGTRSYFGAFMV

SEQ ID NO: 54 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVSDERLVRTRDGTRSYFGAFMV

SEQ ID NO: 55 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVTDERLVRTRDGTRSYFGAFMV

SEQ ID NO: 56 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVTDERLVRTRDGTRSYFGAFMV

SEQ ID NO: 57 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVSDERLVRTRDGTRSYFGAFMV

SEQ ID NO: 58 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDERLVRLRDGTRSYFGAFMV

SEQ ID NO: 59 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDERLVRLRDGTRSYFGAFMV

SEQ ID NO: 60 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDSRLVRLRDGTRSYFGAFMV

SEQ ID NO: 61 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDSRLVRLRDGTRSYFGAFMV

SEQ ID NO: 62 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVTDERLVRLRDGTRSYFGAFMV

SEQ ID NO: 63 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASGITHGLYKRTPRYPEELELLVSQQSPCGRATSSSRVWWDSSFLGGVVHLEAGEKVVVRVSDERLVRTRDGTRSYFGAFMV

SEQ ID NO: 64 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASPITHGLYKRTPRYPEELELLVSQQSPCGRATSSSRVWWDSSFLGGVVHLEAGEKVVVRVSDERLVRTRDGTRSYFGAFMV

SEQ ID NO: 65 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGTITHGLYKRTPRYPEELELLVSQQSPCGRATSSSRVWWDSSFLGGVVHLEAGEKVVVRVSDERLVRTRDGTRSYFGAFMV

SEQ ID NO: 66 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGTITHGLYKRTPRYPEELELLVSQQSPCGRATSSSRVWWDSSFLGGVVHLEAGEKVVVRVTDERLVRTRDGTRSYFGAFMV

SEQ ID NO: 67 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASSITHGLYKRTPRYPEELELLVSQQSPCGRATSSSRVWWDSSFLGGVVHLEAGEKVVVRVSDERLVRTRDGTRSYFGAFMV

SEQ ID NO: 68 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDERLVRTRDGTRSYFGAFMV

SEQ ID NO: 69 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDERLVRTRDGTRSYFGAFMV

SEQ ID NO: 70 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLASGITHGLYKRTPRYPEELELLVSQQSPCGRATSSSRVWWDSSFLGGVVHLEAGEKVVVRVSDERLVRTRDGTRSYFGAFMV

SEQ ID NO: 71 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLASPITHGLYKRTPRYPEELELLVSQQSPCGRATSSSRVWWDSSFLGGVVHLEAGEKVVVRVSDERLVRTRDGTRSYFGAFMV

SEQ ID NO: 72 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGTITHGLYKRTPRYPEELELLVSQQSPCGRATSSSRVWWDSSFLGGVVHLEAGEKVVVRVSDERLVRTRDGTRSYFGAFMV

SEQ ID NO: 73 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLASGITHGLYKRTPRYPEELELLVSQQSPCGRATSSSRVWWDSSFLGGVVHLEAGEKVVVRVSDERLVRTRDGTRSYFGAFMV

SEQ ID NO: 74 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDERLVRTRDGTRSYFGAFMV

SEQ ID NO: 75 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDERLVRTRDGTRSYFGAFMV

SEQ ID NO: 76 - skipped

SEQ ID NO: 77 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDERLVRTRDGTRSYFGAFMV

SEQ ID NO: 78 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGSITHGLYKRTPRYPEELELLVSQQSPCGRATSSSRVWWDSSFLGGVVHLEAGEKVVVRVSDERLVRTRDGTRSYFGAFMV

SEQ ID NO: 79 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLAGSITHGLYKRTPRYPEELELLVSQQSPCGRATSSSRVWWDSSFLGGVVHLEAGEKVVVRVSDERLVRTRDGTRSYFGAFMV

SEQ ID NO: 80 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDSRLVRTRDGTRSYFGAFMV

SEQ ID NO: 81 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDSRLVRTRDGTRSYFGAFMV

SEQ ID NO: 82 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVSDSRLVRTRDGTRSYFGAFMV

SEQ ID NO: 83 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLAGPITHGLYKRTPRYPEELELLVSQQSPCGRATSSSRVWWDSSFLGGVVHLEAGEKVVVRVSDSRLVRTRDGTRSYFGAFMV

SEQ ID NO: 84 - protein, 167 aa, synthetic construct
DGPAGSWEQLIQERRSHEVNPAAHLTGANSSLTGSGGPLLWETNLGLAFLRGLSYHDGALVVTKAGYYYIYSKVQLGGVGCPLGLAGSITHGLYKRTPRYPEELELLVSQQSPCGRATSSSRVWWDSSFLGGVVHLEAGEKVVVRVSDSRLVRTRDGTRSYFGAFMV

SEQ ID NO: 85 - protein, 167 aa, synthetic construct
DGPAGSWEQLIQERRSHEVNPAAHLTGANSSLTGSGGPLLWETELGLAFLRGLSYHDGALVVTKAGYYYIYSKVQLGGVGCPLGLAGSITHGLYKRTPRYPEELELLVSQQSPCGRATSSSRVWWDSSFLGGVVHLEAGEKVVVRVSDSRLVRTRDGTRSYFGAFMV

SEQ ID NO: 86 - protein, 240 aa, synthetic construct
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 87 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 88 - protein, 154 aa, synthetic construct
RRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 89 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGLARTITHGLYKRTPRYPEELELLVSQQSPCGRATSSSRVWWDSSFLGGVVHLEAGEKVVVRVSDERLVRLRDGTRSYFGAFMV

SEQ ID NO: 90 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLRGVGCPLGLARTITHGLYKRTPRYPEELELLVSQQSPCGRATSSSRVWQDSSFLGGVVHLEAGEKVVVRVSDERLVRLLDGTRSYFGAFMV

SEQ ID NO: 91 - protein, 167 aa, synthetic construct
DGPAGSWEQLIQERRSHEVNPTAHLTGANSSLTGSGGPLLWETQLGLAFLRGLSYHDGALVVTKAGYYYIYSKVQLRGVGCPLGLARTITHGLYKRTPRYLEELELLVSQQSPCGRATSSSRVWQDSSFLGGVVHLEAGEKVVVRVSDERLVRLLDGTRSYFGAFMV

SEQ ID NO: 92 - protein, 167 aa, synthetic construct
DGPAGSWEQLIQERRSHEVNPTAHLTGANSSLTGSGGPLLWETQLGLAFLRGLSYHDGALVVTKAGYYYIYSKVQLRGVGCPLGLARTITHGLYKRTPRYLEELELLVSQQSPCGRATSSSRVWQDSSFLGGVVHLEAGEKVVVRVLDERLVRLLDGTRSYFGAFMV

SEQ ID NO: 93 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLAGGITHGLYKRTPRYPEELELLVSQQSPCGRATSSSRVWWDSSFLGGVVHLEAGEKVVVRVTDSRLVRLRDGTRSYFGAFMV